(19) United States
(12) Patent Application Publication
(10) Pub. No.: US 2026/0232384 A1
(43) Pub. Date: Aug. 13, 2026

(54) THREE DIMENSIONAL IMAGING FOR SURGERY

(71) Applicant: Michael S. Berlin, Las Vegas, NV (US)

(72) Inventor: Michael S. Berlin, Las Vegas, NV (US)

(21) Appl. No.: 19/153,845

(22) PCT Filed: Feb. 20, 2024
(86) PCT No.: PCT/US2024/016529

§ 371 Date: Aug. 5, 2025

Related U.S. Application Data
(60) Provisional application No. 63/485,808, filed on Feb. 17, 2023.
(60) Provisional application No. 63/597,240, filed on Nov. 8, 2023.

(51) Int. Cl. A61B 34/20; A61B 3/00; A61B 90/00; A61M 25/01

(52) U.S. Cl. CPC A61B 34/20; A61B 3/0025; A61B 2034/2065; A61B 2090/367; A61M 2025/0166

(57) ABSTRACT

In some embodiments, an imager is coupled to a sensor to measure one or more of a position or an orientation of a measurement or imaging beam of the imager while the imager acquires image data. A processor is configured to receive the image data and construct an image such as a 3D image in response to the image data and the sensor data. In some embodiments, the imager is configured to emit a beam of imaging energy, and movement of the imager is used to construct the image such as a 3D image. In some embodiments, movement that occurs during the acquisition of the image data allows a 3D image to be constructed without a scanner, which can decrease the complexity of the imaging apparatus. Alternatively or in combination, the imager may comprise a scanner or beam former, and the movement data can be used to improve image quality.

### RELATED APPLICATIONS

This application is a U.S. National Phase based on and claiming priority to PCT/US24/16529, filed Feb. 20, 2024, which claims the benefit under 35 U.S.C. §119(e) of U.S. Provisional Patent Application No. 63/597,240, filed Nov. 8, 2023, and of U.S. Provisional Patent Application No. 63/485,808, filed Feb. 17, 2023, the disclosures of which are incorporated, in their entirety, by this reference.

### BACKGROUND

Prior approaches to imaging tissue with imaging such as optical coherence tomography (OCT) can be less than ideal in at least some respects. Work in relation to the present disclosure suggests that at least some of the prior approaches to imaging object such as tissue can be somewhat more complex than would be ideal. Although imaging approaches such as OCT, ultrasound and photoacoustic imaging have found application in many fields such as imaging tissue for diagnosis and surgery, at least some of the prior approaches are less than ideally suited for integration with a surgical system, for example. With some prior imaging systems, a series of A-scans can be used to generate images such as B-scan and 3D images such as tomographic images. However, unintended movement of an imaging beam can make the construction of images from data sourced from imaging components to which unintended movement is inherent more difficult than would be ideal, in at least some instances. Also, at least some of the prior imaging systems rely on a scanning device to move the imaging beam can be more complex than ideal in at least some instances. In imaging systems in which data is acquired with a hand held probe, one source of potential movement is human tremor, for example when a surgical endoscope is held or manipulated by a surgeon.

Another example is movement such as resonance movement associated with mechanical devices such as surgical robots. Also, work in relation to the present disclosure suggests that human tremor from a human operator controlled robotic arm can result in at least some movements of the robotic arm that are related to the tremor of the operator. Work in relation to the present disclosure suggests that at least some of the prior approaches have less than ideally addressed movement such as movement related to human tremor and mechanical resonance.

An example of surgeries which can be treated with improved imaging are ophthalmic surgeries, and specifically glaucoma surgeries, for example. Although some treatments can be successful, the prior approaches to treating glaucoma can be less than ideal in a least some respects. One approach to treat glaucoma is with minimally invasive glaucoma surgery (“MIGS”). With canal based MIGS, a small opening is created through the trabecular meshwork to allow fluid to drain into Schlemm's canal. These openings can be created in many ways, for example with implants or lasers such as femto second lasers. One approach has been to use excimer laser trabeculostomy (“ELT”), in which an ultraviolet laser such as an excimer laser is used to ablate one or more openings through the trabecular meshwork into Schlemm's canal. Another approach has been to place an implant that extends through the trabecular meshwork into Schlemm's canal.

One potentially challenging aspect of canal based MIGS procedures is alignment of a surgical instrument with Schlemm's canal, which can be approximately 200 micrometers (“μm”) to 400 μm in height. In some instances, Schlemm's canal may not be readily visible, in which case the surgeon must try to estimate the location of Schlemm's canal, which can be challenging and less than ideally accurate in at least some instances. With some implantation procedures, inaccurate assessment of the location of Schlemm's canal may lead to several less than ideal situations such as the implant not being appropriately positioned in the canal, tearing of the trabecular meshwork, and in some instances malpositioning of an implant can result in it becoming subsequently dislodged, for example.

Although OCT, ultrasound, and photoacoustic imaging have been proposed as an approach to imaging Schlemm's canal, work in relation to the present disclosure suggests that prior approaches to imaging Schlemm's canal may be less than ideal in at least some instances. For example, the imaging systems may be somewhat more complex than ideal, and the prior systems may not adequately address movement such as human tremor or movement of the in vivo tissue itself. In at least some instances, tissue movement may be related to its inherent pulsation due to cardiac cyclic filling and emptying of vascular capillary networks of an organ, which can result in movement of the tissue, and the prior approaches can be less than ideal in addressing the pulsatile movement of the tissue.

In light of the above, it would be beneficial to have improved OCT methods and apparatus, for example to facilitate the creation of openings in Schlemm's canal and to facilitate the placement of implants.

### SUMMARY

In some embodiments, an imager is configured to generate a beam of imaging energy and the imager is coupled to a sensor to measure one or more of a position or an orientation of the imaging beam while the imager acquires image data. The sensor may comprise one or more of a position sensor, an orientation sensor, an accelerometer, or an image sensor. A processor is configured to receive the image data and construct an image such as a 3D image in response to the acquired image data and the acquired sensor data. In some embodiments, the imager is configured to emit the beam of imaging energy, and movement data of the imaging beam is used to construct the image such as a 3D image. In some embodiments, this movement which occurs during the acquisition of the image data allows a 3D image to be constructed without the use a scanner, which can decrease the complexity of the imaging apparatus. Alternatively or in combination, the imager may comprise a scanner or beam former, and the movement data can be used to improve the quality of the 3D images.

In some embodiments, the concurrent position and orientation data of the imaging device is used to determine the position and orientation of the imaging beam while the image data is acquired, and the position and orientation data of the imaging beam is used to construct a 3D image of the tissue. In some embodiments, the imaging beam is configured to acquire A-scan data and a position and orientation of imaging beam is acquired for each of a plurality of A-scans, and the position and orientation data is combined with the plurality of A-scans to generate the 3D image.

In some embodiments, a distal end of an imaging channel is located proximally to a distal end of a treatment channel in order to view the distal end of the treatment channel with the imaging channel, such as a 3D imaging channel. In some embodiments, tissue and one or more of a treatment fiber or an implant are visible in 3D images generated by the imaging channel in order to view the relationship of the tip of the treatment channel or implant and the tissue, which can facilitate the placement of treatment. In some embodiments, the imaging channel comprises an optical fiber comprising a distal end, which is located proximally to a distal end of a treatment channel, such as a treatment optical fiber or an implant, in order to view a relationship between tissue and the distal end of the optical fiber or the implant.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a schematic sectional view of an eye illustrating anatomical structures, in accordance with some embodiments;

FIG. 2 shows a perspective partial view of the anatomy adjacent to the anterior chamber of an eye, in accordance with some embodiments;

FIG. 3 shows a schematic sectional view of an eye illustrating a fiber optic probe and imaging probe crossing the anterior chamber from a corneal limbal paracentesis site toward the trabecular meshwork in the anterior chamber of the eye, in accordance with some embodiments;

FIG. 4A shows a partial schematic view of the anatomy of the anterior chamber angle of an eye showing Schlemm's canal, the scleral spur and Schwalbe's line, in accordance with some embodiments;

FIG. 4B shows a partial view of the anatomy of an eye and is representative of an image obtained with an endoscope or other imaging system from the viewpoint of within an eye, in accordance with some embodiments;

FIG. 5A shows a components of OCT imaging system and treatment probe for imaging and treating tissue of the eye, in accordance with some embodiments;

FIG. 5B shows an ultrasound imaging system and treatment probe for imaging and treating tissue of the eye, in accordance with some embodiments;

FIG. 5C shows a photoacoustic imaging system and treatment probe for imaging and treating tissue of the eye, in accordance with some embodiments;

FIG. 6A shows a scanning OCT imaging system and treatment probe for imaging and treating tissue of the eye, in accordance with some embodiments;

FIG. 6B shows an ultrasound imaging system and treatment probe for imaging and treating tissue of the eye, in accordance with some embodiments;

FIG. 6C shows a scanning photoacoustic imaging system and treatment probe for imaging and treating tissue of the eye, in accordance with some embodiments;

FIG. 7 shows an apparatus for eye surgery, in accordance with some embodiments;

FIG. 8 shows an augmented image comprising an optical operating microscope view and one or more of a 2D image, a 3D image, or a model overlaid with treatment site markers, in accordance with some embodiments;

FIG. 9 shows movement of a probe and corresponding positions of a measurement or imaging beam, in accordance with some embodiments;

FIG. 10 shows a probe comprising an orientation sensor pivoting about an opening, in accordance with some embodiments;

FIG. 11 shows a probe comprising an endoscope configured to determine locations of a measurement or imaging beam of a 3D imager, in accordance with some embodiments;

FIG. 12 shows a probe comprising treatment channel, a 3D imager and an endoscope to determine a location of the measurement or imaging beam of the 3D imager, in accordance with some embodiments;

FIG. 13A shows a probe comprising a treatment channel, and 2D and 3D imaging components comprising an overlapping optical path, in accordance with some embodiments;

FIG. 13B shows a probe comprising a treatment channel and a 3D imaging optical fiber and a plurality of 2D imaging optical fibers, in accordance with some embodiments;

FIG. 14A shows an endoscope image comprising one or more tissue structures and a location of a measurement or imaging beam at a first time, in accordance with some embodiments;

FIG. 14B shows an endoscope image comprising one or more tissue structures and a location of a measurement or imaging beam at a second time, in accordance with some embodiments;

FIG. 14C shows an image displacement vector and corresponding measurement locations, in accordance with some embodiments;

FIG. 15 shows movement of a probe and corresponding positions of a measurement or imaging beam, in accordance with some embodiments;

FIG. 16 shows a method of imaging tissue, in accordance with some embodiments; and

FIG. 17 shows a femtosecond laser and OCT system comprising a sensor, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The methods and systems disclosed herein can generate images of the eye such as 3D OCT images of the eye and the anatomy of the eye to allow more ophthalmic surgeons to successfully image the eye and to perform MIGS procedures, such as the placement of implants and the creation of openings in the trabecular meshwork, for example with one or more of tissue manipulation, incision, or ablation. The presently disclosed methods and systems are well suited for use with surgical instruments such as hand held instruments or robotic manipulators. For example, the disclosed methods and apparatus can allow for surgeries to more uniformly and consistently create openings to enable improved outflow of aqueous fluid from the eye's anterior chamber into Schlemm's canal, for example. In addition, the disclosed system and methods can lead to improved surgical outcomes, by allowing surgeons to generate an image of the eye such as a 3D image and identify target locations for openings into Schlemm's canal intended to increase outflow. In some cases, a target location may include a volume, surface or layer of a tissue, or a 3D position at a tissue, for example of the trabecular meshwork, the juxtacanalicular trabecular meshwork (JCTM), the inner wall of the Schlemm's canal, the outer wall of the Schlemm's canal, the sclera, or desired combinations thereof.

The presently disclosed methods and apparatus may include the combination of an imaging device, imaging sensors, and position and orientation sensors which enable real-time display of 2D and 3D images and models to be concurrently viewed by the surgeon. The position and orientation sensor data can be combined with image data to provide improved images. The real-time display of 2D and 3D images and models may include 2D and 3D images and models that are updated during procedures with decreased latencies. In some embodiments, the real-time augmented display shows images, including video, of 2D and 3D images and models as events are happening. These augmented images and models enable the surgeon to view, target and treat locations within an eye which may not be readily visualized using an operating microscope or camera alone, due to their location within the eye at sites in which total internal reflections precludes their visualization in the microscope image, unaided. Such structures include the trabecular meshwork and Schlemm's canal.

In some embodiments, the imager such as a 3D imager comprises one or more of an OCT system, an ultrasound system, or a photoacoustic system, and includes one or more emitters and associated sensors located on a handpiece of the probe.

The images and models generated by the imaging system and position and orientation sensor data can be presented to the surgeon in many ways. For example, the images and models can be superimposed on an image viewed via a monitor or similar viewing devices, such as augmented reality glasses, or goggles or virtual reality glasses or goggles. In some embodiments, a real-time image from an imager is presented on a monocular or a binocular heads up display with an optical image of the eye from a microscope, such as an operating microscope, which allows the surgeon to view both the optical image and generated 2D and 3D images and models while looking into the microscope or at an image generated with a microscope. Additional information can also be provided to the surgeon, such as virtual images and models of otherwise non-visible structures and one or more symbols to indicate both distances and movement, such as from a probe tip to trabecular meshwork to Schlemm's canal.

In some embodiments, the imaging system can be used to identify collector channels of the eye and enable the surgeon to identify sites by these target locations (e.g. by using a graphical visual element such as a treatment reference marker to identify a target location) displayed to the user to assist in the creation of openings at appropriate locations in the trabecular meshwork to increase flow. In some embodiments, images as such images as collector channel images can be obtained pre-operatively, such as with OCT imaging, and superimposed on images of the eye to allow the surgeon to identify the locations of collector channels. In some embodiments, image analysis algorithms are applied to images to recognize anatomical features within the eye during surgery and a heads-up display can augment the real-time 2D and 3D images and models with recognized features, guides, locations, markers, and the like to assist the surgeon in performing the surgery.

Such displays can be coupled to the operating microscope in order to present monocular or binocular virtual and/or augmented 2D and 3D images and models from a display which is visually combined with binocular real optical images of the eye, for example. The methods and apparatus disclosed herein are well suited for utilization with ELT surgery and with an implant device such as stent surgeries which provide openings to drain fluid from the eye. However, the provided system and methods can also be applied to various other surgical procedures where fiberoptic-based imaging may be utilized, e.g. any and all surgeries using an endoscope.

Although specific reference is made to the treatment of glaucoma using excimer laser trabeculostomy (“ELT”), the methods and systems disclosed herein can be used with many other types of surgeries. For example, the embodiments disclosed herein can be used with other surgical procedures, including endoscopic procedures relating to orthopedic, neurosurgical, neurologic, ear nose and throat (ENT), abdominal, thoracic, cardiovascular, epicardial, endocardial, and other applications to name a few. The presently disclosed methods and apparatus can utilize in-situ imaging to improve targeting accuracy and provide virtual visualization for enabling surgeons to perform procedures in regions that may not be readily visualized either microscopically or endoscopically, such as images obtained with operating microscopes, goniolenses and slit lamps. Such applications include any endoscopic procedure in which virtual visualization is augmented to images of real objects to assist surgical accuracy in 3-dimensional space, one example of which is an endovascular procedure in which the vessel curves or bends. In some cases, an in-situ imaging system is carried by or with the treatment probe and captures images from the treatment site or along the path to the treatment site to allow the surgeon to see actual anatomical features.

Some aspects may also be used to treat and modify other organs such as brain, heart, lungs, intestines, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone, as well as body lumens and passages such as the sinuses, ureter, colon, esophagus, lung passages, blood vessels, and throat. For example, the devices disclosed herein may be inserted through an existing body lumen or inserted through an opening created in body tissue.

In some embodiments, tissue movement is measured and used to construct one or more images as described herein. In some embodiments, the imaged tissue comprises a tissue of a pulsatile flow fluid system such as vascular tissue or trabecular meshwork and collector channels, as described herein. In some embodiments, the collector channels and Schlemm's canal are coupled to the vascular system with pulsatile flow through the imaged ocular tissue. In some embodiments, a pulsatile pump, e.g. the heart, results in periodic movement of tissue related to the pulsatile flow. In some embodiments, the pulsatile flow may result in a “jellyfish” like movement of the living tissues. In some embodiments, this pulsatile movement generates image displacement with images acquired from imaging sensors as described herein. In some embodiments, the displacement can be monitored and compensated, e.g. by sensing the cardiac cycle pulsation, and compensating for the displacement in response to the sensors configured to detect the cardiac cycle, for example.

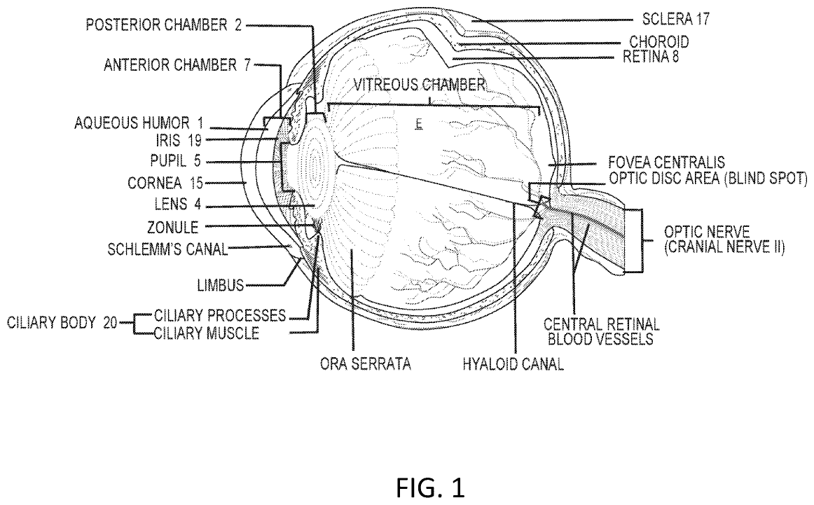

With reference to FIG. 1, in order to appreciate the described embodiments, a brief overview of the anatomy of the eye E is provided. As schematically shown in FIG. 1, the outer layer of the eye includes a sclera 17. The cornea 15 is a transparent tissue which enables light to enter the eye. An anterior chamber 7 is located between the cornea 15 and an iris 19. The anterior chamber 7 contains a constantly flowing clear fluid called aqueous humor 1. The crystalline lens 4 is supported and moved within the eye by fiber zonules, which are connected to the ciliary body 20. The iris 19 is attached circumferentially to the scleral spur and includes a central pupil 5. The diameter of the pupil 5 controls the amount of light passing through the lens 4 to the retina 8. A posterior chamber 2 is located between the iris 19 and the ciliary body 20.

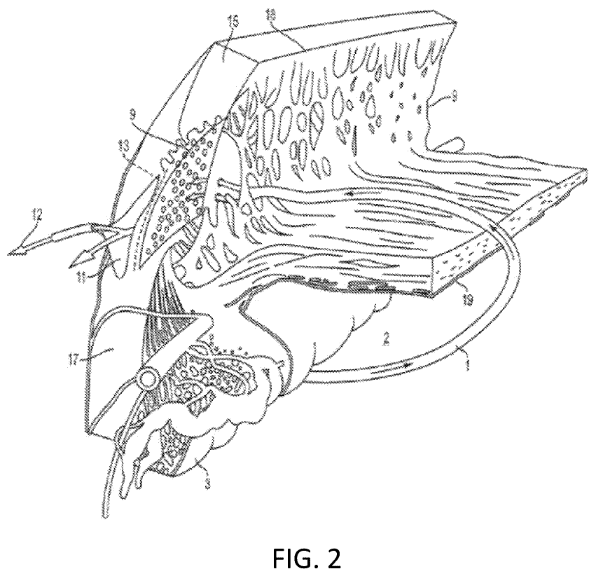

As shown in FIG. 2 the anatomy of the eye further includes a trabecular meshwork (TM) 9, a triangular band of spongy tissue within the eye that lies anterior to the iris 19 insertion to the scleral spur. The mobile trabecular meshwork continuously varies in shape and is microscopic in size. It is generally triangular in cross-section, varying in thickness from about 100-200 μm. It is made up of different fibrous layers having micron-sized pores forming fluid pathways for the egress of aqueous humor from the anterior chamber. The trabecular meshwork 9 has been measured to about a thickness of about 100 μm at its anterior edge, Schwalbe's line 18, at the approximate juncture of the cornea 15 and sclera 17.

The trabecular meshwork widens to about 200 μm at its base where it and iris 19 attach to the scleral spur. The height of the trabecular meshwork can be about 400 μm. The passageways through the pores in trabecular meshwork 9 lead through a very thin, porous tissue called the juxtacanalicular trabecular meshwork 13, which in turn abuts the inner wall of a vascular structure, Schlemm's canal 11. The height of Schlemm's canal can be about 200 μm, or about half the height of the trabecular meshwork. Schlemm's canal (SC) 11 is filled with a mixture of aqueous humor and blood components and connects to a series of collector channels (CCs) 12 that drain the aqueous humor into the venous system. Because aqueous humor 1 is constantly produced by the ciliary body and flows through the pupil into the anterior chamber from which it passes through pores in the TM and JCTM into the SC, collector channels and aqueous veins, any obstruction in the trabecular meshwork, the juxtacanalicular trabecular meshwork, or Schlemm's canal, prevents the aqueous humor from readily egressing from the anterior eye chamber. As the eye is essentially a thick wall enclosed globe, inflow with obstructed outflow can result in an elevation of intraocular pressure within the eye. Increased intraocular pressure can lead to damage of the retina and optic nerve, and thereby cause eventual blindness.

The obstruction of the aqueous humor outflow, which occurs in most open angle glaucoma (i.e., glaucoma characterized by gonioscopically readily visible trabecular meshwork), is typically localized to the region of the juxtacanalicular trabecular meshwork (JCTM) 13, located between the trabecular meshwork 9 and Schlemm's canal 11, and, more specifically, the inner wall of Schlemm's canal.

When an obstruction develops, for example, at the juxtacanalicular trabecular meshwork 13, intraocular pressure gradually increases over time. Therefore, a goal of current glaucoma treatment methods is to prevent optic nerve damage by lowering or delaying the progressive and chronic elevation of intraocular pressure.

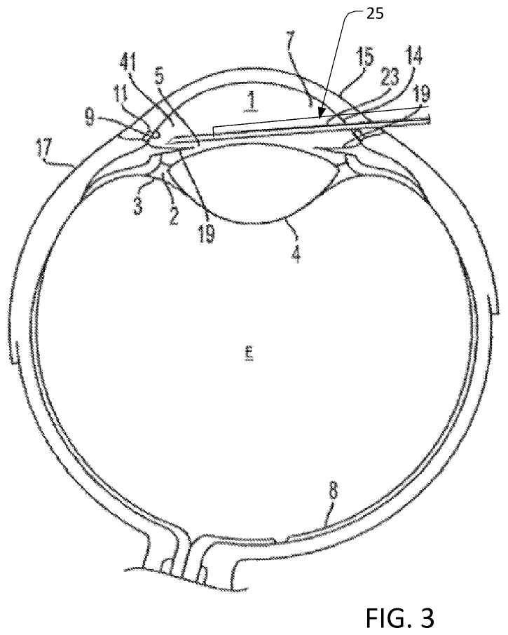

With reference to FIG. 3, a side sectional view of the interior anatomy of a human eye E is shown with a treatment probe comprising fiber-optic probe 23, and an imaging probe such as a 2D or 3D imaging device coupled to or incorporated with the probe inserted into the eye in accordance with some embodiments. A small self-sealing paracentesis incision 14 is created in the cornea 15. The anterior chamber can be stabilized with either a chamber maintainer using liquid flows or a viscoelastic agent. Fiber-optic treatment probe 23 and the imaging probe 25 such as a 2D or 3D imaging device can then be positioned and advanced through the incision 14 into the anterior chamber 7 until a distal end of the fiber-optic treatment probe 23 contacts and slightly compresses the desired target TM tissues. The imaging probe 25 may comprise any suitable imaging probe such as an optical imaging probe, an endoscope, an OCT imaging probe, an ultrasound imaging probe, or a photoacoustic imaging probe as described herein, for example.

In some embodiments, the distal tip of the treatment probe such as fiber-optic probe 23 extends beyond the distal tip of the imaging probe 23 to image the distal tip of the treatment probe with the imaging probe. Although reference is made to separate probes, in some embodiments, the probes are combined into a single probe to perform imaging and treatment, for example with a housing over both a treatment channel and an imaging channel as described herein. In some embodiments, the imaging channel and treatment channel are configured to image concurrently the target tissue region the treatment probe tip, so that the surgeon can see where the treatment will occur, for example with the treatment probe tip extending beyond the imaging probe tip to the view treatment probe with the imaging probe. This configuration with the treatment tip extending beyond the imaging tip can be used with laser treatment probe tips, stent treatment probe tips, tissue manipulator tips, and the tips of incision instruments, for example.

In some embodiments, the distal end of the treatment channel, e.g. treatment fiber in the case of a laser, is offset by several mm in front of the viewing fiber so as to visualize the treatment channel with the imager. In some embodiments, the treatment channel comprises an implant, which can be visualized with the imager such as a 3D imager, in order to place the implant.

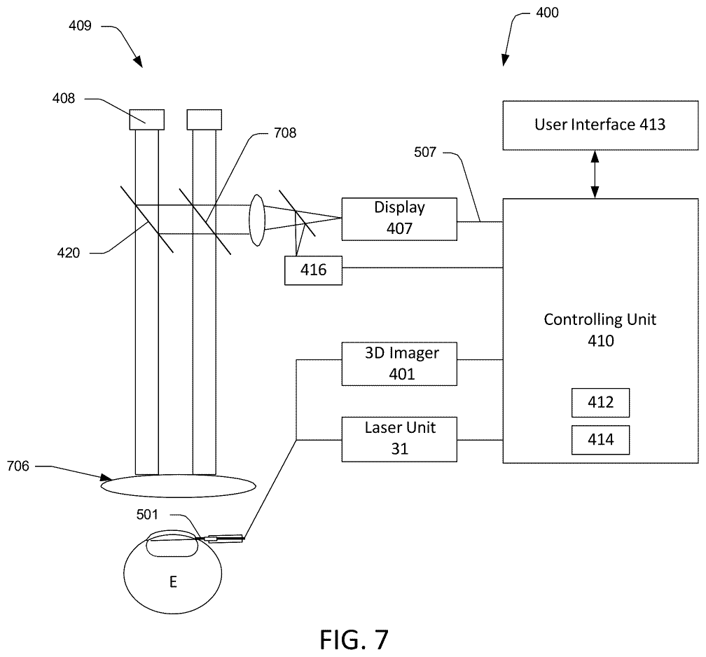

Photoablative laser energy produced by laser unit 31 (shown in FIG. 7) is delivered from the distal end of fiber-optic probe 23 in contact with the tissue to be excised. The tissue to be excised may include the trabecular meshwork 9, the juxtacanalicular trabecular meshwork 13 and an inner wall of Schlemm's canal 11. An aperture in the proximal inner wall of Schlemm's canal 11 is created in a manner which does not perforate the distal outer wall of Schlemm's canal. In some embodiments, additional apertures are created in the target tissues. Thus, the resultant aperture or apertures are effective to restore relatively normal rates of drainage of aqueous humor. The photoablative laser energy may comprise one or more types of laser energy, such as visible, ultraviolet, near infrared, or infrared laser energy, and combinations thereof. In some embodiments, the laser energy comprises 308 nm laser energy from a Xenon Chloride excimer laser. The laser may comprise pulsed energy or substantially continuous energy, for example. In embodiments, the laser energy delivered from the probe comprises femto-second or pico-second laser energy, for example.

The fiber-optic probe 23 may comprise an optical fiber or a plurality of optical fibers encapsulated by an encapsulating sheath. In some embodiments, the diameter of a single treatment optical fiber should be sufficiently large to transmit sufficient light energy to effectively result in excision such as photoablation of target tissues. In some embodiments, the imaging optical fiber diameter is in a range from about 4-6 μm. A single optical fiber or a plurality of optical fibers can be used in a bundle of a diameter ranging from about 100 μm to about 1000 μm, for example. The optical fiber core and cladding can be encased within an outer metal sleeve, or shield. In some embodiments the sleeve is fashioned from stainless steel. In some embodiments, the outer diameter of sleeve is less than about 100 μm. In some embodiments, the diameter can be as small as 100 μm, as where smaller optical fibers are implemented with laser delivery systems. In some cases, the optical fiber may have a diameter of about 200 μm and the fiber-optic probe 23 may have a greater diameter such as 500 μm to encapsulate one or more optical fibers. In some embodiments, the sleeve can be flexible so that it can be bent or angled.

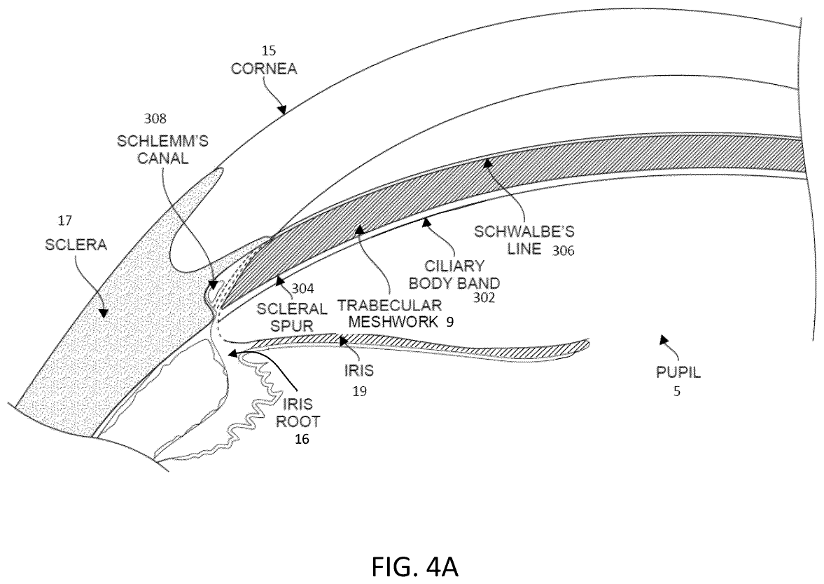

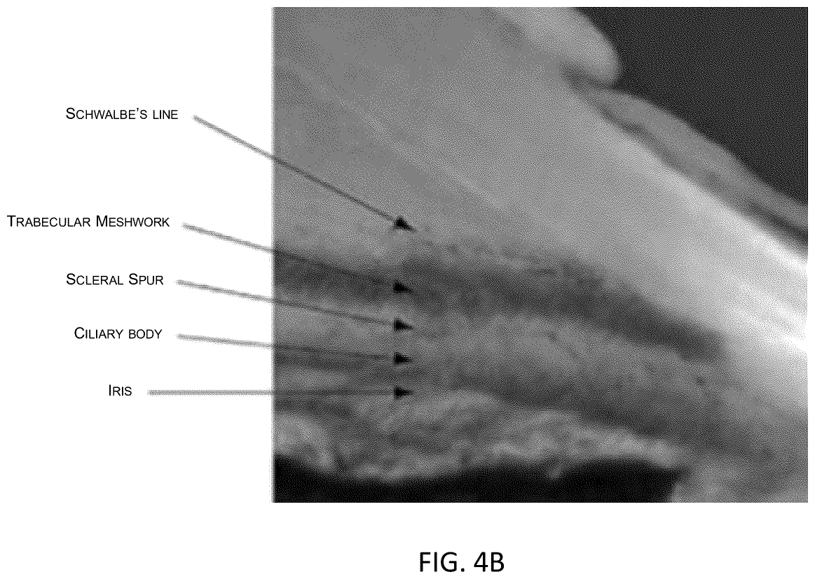

FIGS. 4A and 4B show interior structures of the eye visible with an imager, such as the OCT, ultrasound, and photoacoustic devices and systems described herein, and these structures may also be viewed with one or more of a goniolens, a slit lamp or a microscope, for example. Structures visible with the 3D imager with an ab interno approach as described herein include the ciliary body band 302 and the scleral spur 304. In some embodiments, Schwalbe's line 306 can be viewed with the 3D imager or a portion of the 3D imager, such as the emitter, inserted into the eye. In some embodiments, Schlemm's canal 308 can be seen in the image, depending on the intraocular pressure of the eye during surgery. The methods and apparatus disclosed herein can be well suited for identifying or estimated the locations structures of the eye that may not be readily visible with simple camera images, such as those from a camera optically coupled to an endoscope inserted into the eye.

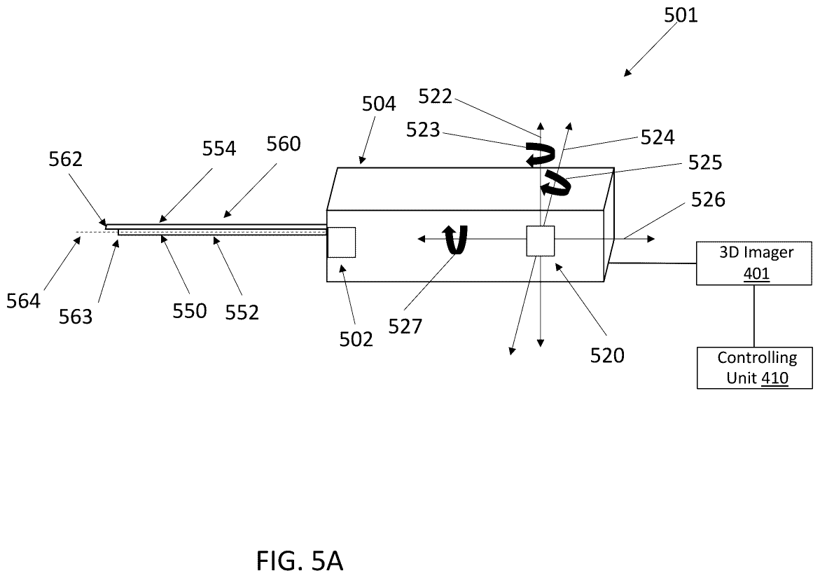

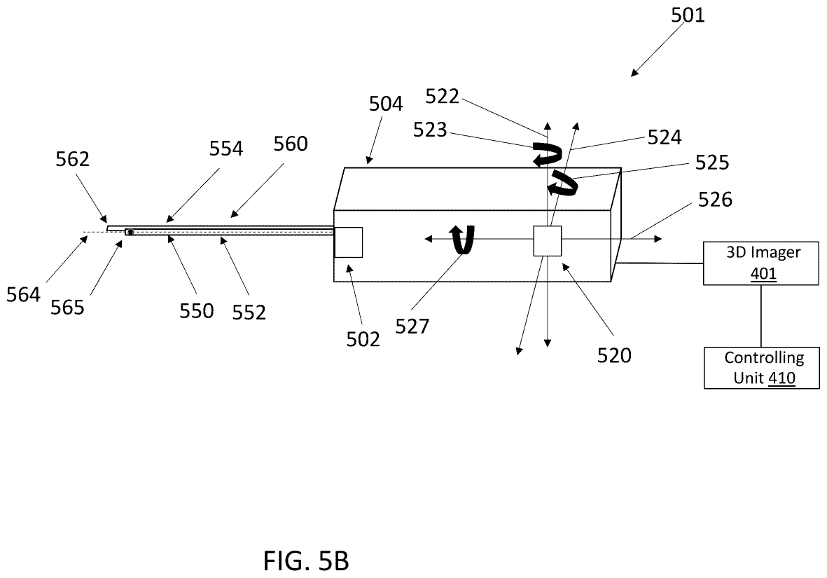

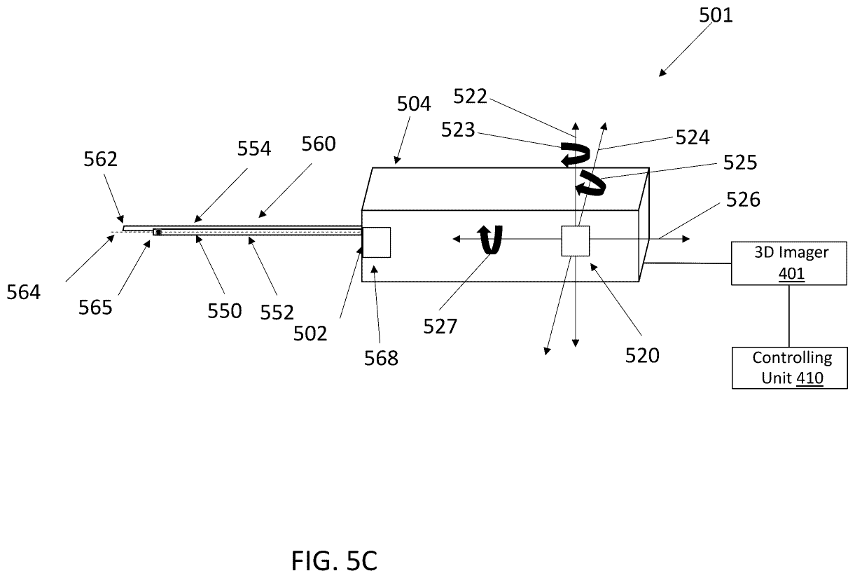

With reference to FIG. 5A an example probe apparatus 501 and imaging device 502 are illustrated in accordance with some embodiments. The imaging device 502 may comprise an OCT imaging device. Portions of the imaging device 502 may be contained within a housing 504, such as a handheld housing, such as the interferometer portions of the OCT imager, including the OCT light source, beam splitter, and reference and sample a detector such as an array of detector elements. Alternatively or in combination, portions of the imaging device may be located externally to the handheld housing, such as the interferometer portions of the OCT imager, including the OCT light source, beam splitter, and reference and sample a detector such as an array of detector elements. In some embodiments, one or more channels 550 may extend along a length of the probe 560.

In some embodiments, the one or more channels 550 comprises an imaging channel 552 and a treatment channel 554. In some embodiments, the imaging channel 552 comprises an optical fiber to transmit and receive the OCT measurement or imaging beam. In some embodiments, the treatment channel 554 comprises one or more optical fibers to deliver treatment light energy to the eye, or a channel to deliver an implant to the eye. Although reference is made to separate imaging and treatment channels, in some embodiments the two channels are combined into a single channel, for example when a single optical fiber is used for the OCT measurement or imaging beam and the laser treatment beam in a multiplexed configuration, for example.

The OCT imaging device may comprise any suitable OCT imaging device, such as a broad spectrum imaging device with a movable mirror, a Fourier domain imaging device, a spectral domain OCT imaging device, or a swept source OCT imaging device, for example, as will be understood by one of ordinary skill in the art.

The probe apparatus 501 may comprise a movement sensor 520 that measures the movements of the probe apparatus 501 in three degrees of translation and three degrees of rotation. For example, the sensor 520, which may be referred to as a movement sensor, may measure movements in three degrees of translation and three degrees of rotation. The three degrees of translation may be three orthogonal axis, such as an x-axis 522, y-axis 524, and z-axis 526. The z-axis may be aligned with or parallel to the imaging and/or treatment axis 564 of the probe 560 while the x-axis and y-axis are in a plane perpendicular to the z-axis. The three degrees of rotation may be about the three orthogonal axis, such as a first degree of rotation 523 about the x-axis 522, a second degree of rotation 525 about the y-axis 525, and a third degree of rotation 527 about the z-axis.

The sensor 520 may comprise any suitable sensor, such as one or more of an accelerometer, a Micro-Electro-Mechanical System (“MEMS”) accelerometer, an inertial sensor, a magnetic field sensor, a gyroscope, a gyrocompass, an inertial measurement unit (IMU). The sensor may be configured to measure orientation and acceleration along 3 or more axes, for example a 3 axis accelerometer configured to measure the position and orientation of the probe, for example.

The treatment probe 560 may comprise any suitable length between the distal tip 562 and housing 504 of the handpiece, and may be between 2 mm and 50 mm long, such as between 2.5 mm and 40 mm long, for example. One or more components of the imaging device 502, such as an imaging sensor, may be located between 2 mm and 50 mm from the distal tip 562 of the treatment probe, such as between 2.5 mm and 30 mm from the tip of the treatment probe. In some embodiments, one or more components of the imaging device 502 is located within a housing 504 of a handpiece of the treatment apparatus 501. Alternatively or in combination, one or more components of the 3D imaging device 502 can be located away from the treatment probe 560 and the handpiece comprising housing 504, for example with in a console of a surgical imaging system as described herein. The one or more channels 550 may include an optical fiber for transmitting light out the tip 562 of the probe.

In some embodiments, the imaging channel 552 extends to a distal tip 563, which is offset by an axial distance from the distal tip 562 probe 560 comprising treatment channel 554, in order to simultaneously image the distal tip 562 and tissue with the imaging channel 552. In some embodiments, the distal tip 562 of the probe 560 comprising treatment channel 554 extends a greater distance distally than the distal tip 563 of the imaging channel 552, such as a 3D imaging channel, in order to simultaneously image tissue and the distal tip 562 with energy emitted from the tip 563 of the imaging channel 552. In some embodiments, the distance between the distal tip 562 of probe 560 comprising treatment channel 554 is within a range from 0 mm to 20 mm, and can be within a range from 1 mm to 10 mm, or 2 mm to 8 mm, for example.

In some embodiments, the distal end of the probe 560 and the one or more channels 550 may be formed with an inclined surface having an angle relative to the longitudinal axis of the probe 560 to compress the trabecular meshwork, which may be inclined relative to the elongate axis of the probe.

The imaging channel 552 and the treatment channel 554 can be arranged in many ways. For example, the imaging channel 552 can be located below treatment channel 554 as shown. Alternatively, the imaging channel 552 can be located above the treatment channel 554, for example.

In some embodiments, a single optical fiber may be used for the treatment channel or the imaging channel, for example. In some embodiments, a bundle of optical fibers, such as two or more fibers could be used with the disclosed systems and methods. In some examples, the treatment channel 554 of probe 560 comprises a bundle of optical fibers that each have a distal end at or near the distal end of the probe apparatus 501.

In some embodiments, the 3D image is constructed in response to the image displacement, which can be related to varied orientation of one or more fibers, such as a single fiber or one or more fibers of a multifiber array. The displacement of the images can be used to generate depth data. The image displacement can be related to movement of the probe, or movement of the tissue, such as pulsatile movement as described herein.

In some embodiments, the OCT system 502 images the eye with OCT A-scans. During treatment, such as during imaging of the patient's eyes, the probe apparatus 501 may move in translation and rotation causing the OCT system 502 to generate A-scans from different positions and orientations. The movement can come from many sources, such as human tremor or resonance modes from a robotic arm, for example. The position and orientation data can be combined with the OCT data to construct images in response to the position and orientation of the probe.

The probe apparatus 501 may be coupled in electronic communication with a 3D imager 401 and a controlling unit 410, both of which are also described in further detail herein, such as with respect to FIG. 7. The 3D imager 401 may comprise any suitable imager to image the tissue, such as one or more of an optical imager, an optical coherence tomography (OCT) imager, an ultrasound (US) imager or a photoacoustic imager. In some embodiments, a sensor is coupled to the imager to acquire sensor data related to one or more of a position or an orientation of the imager. In some embodiments, a processor is coupled to the imager and the sensor to acquire image data and sensor data, and the processor is configured with instructions to construct a 3D image of the tissue in response to the imager data and the sensor data.

While the system can be configured in many ways in some embodiments, the imager comprises the sensor, in which the sensor configured to generate movement data, and the processor configured to reconstruct the 3D image in response to the movement data.

While the imager may comprise any imager described herein, in some embodiments, 3D imager 401 comprises the optical imager, in which the optical imager comprises one or more of an endoscope, a microscope, a stereoscopic microscope, a stereoscopic endoscope. In some embodiments the optical imager is configured to capture images related to movement, and the processor is configured to generate movement data and construct the 3D images in response to the movement data as described herein. The optical imager can be configured to generate 3D image data, such as with stereophotogrammetry with images captured from sensor arrays as described herein.

During operation, the imaging device 502, such as an OCT imaging device, may generate imaging data, such as A-scans while the sensor 520 measures the movement of the imaging device in both translation and rotation. The imaging data and movement data may be time-stamped or otherwise correlated with each other such that the position and orientation of the probe apparatus 501 may be associated with each A-scan of the OCT scanner. The imaging data and the movement data may be processed by the 3D imager 401 and/or the controlling unit 410 in order to build a 3D model. The 3D model may be built based on data acquired as the OCT imaging device moves and captures data of different portions of the patient's eye. The 3D imager 401 and/or the controlling unit 410 may then assemble a 3D model by placing each A-scan in spatial orientation with respect to each other A-scan, based on the sensor movement data.

In some embodiments, the imaging device and the sensor may be coupled to a processor configured to acquire image data from the image sensor and sensor data, such as movement data, from the sensor and construct one or more of a 2D image, a 3D image or a 3D model based on or in response to the image data and the sensor data. The imaging device may have an axis, such as a z-axis along which it emits a beam of imaging energy, such a coherent light energy. The axis of the beam may be aligned with respect to or parallel to at least one of axes 522, 524, 526 of the sensor for measuring one or more of the position or the orientation beam of imaging energy. The sensor may be configured to measure a movement of the imaging device and/or imaging beam in a direction along the axis of the beam corresponding to a movement of the measurement or imaging beam along the axis. The processor may then construct a 2D image, a 3D image or a 3D model, such as a tissue model, in response to the movement of the measurement or imaging beam along the axis.

In some embodiments, the sensor is configured to measure a movement of the imager transverse to the axis of the measurement or imaging beam axis 564. Such movement may comprise movement in a plane that is substantially perpendicular to the axis of the measurement or imaging beam (e.g. to within about 10 degrees of perpendicular) and, in some embodiments, also perpendicular to the z-axis, such as a plane defined by the x-axis 522 and y-axis 524. The processor may then construct 2D images, 3D images, or a 3D model such as a tissue model in response to the movement of the measurement or imaging beam transverse to the axis. In some embodiments, the movement data comprises rotational movement data, such as rotation about one or more of the x-axis 522, the y-axis 524, or the z-axis 526. In some embodiments, rotation of the probe about the elongate axis of the probe such as rotation about z-axis 526 results of rotation of the imaging channel 552 and the treatment channel 554 relative to each other, and the processor can be configured with instructions to construct the image such as a 3D image in response to rotation about the elongate axis of the probe.

In some embodiments, the movement data may include data of the rotation of the probe and attached imaging device. In some embodiments, the movement data may include data of the translation of the probe and attached imaging device.

In some embodiments, the processor or processors located in one or more of the 3D imager 401 and the controlling unit 410 may store data of the positions and orientations of the imager while the beam of imaging energy is directed toward the tissue and construct the 2D image, 3D image, or a 3D model from the plurality of positions and orientations of the sensor.

As discussed herein, the imager may generate A-scans with the measurement or imaging beam and the processor may determine a position of the measurement or imaging beam for each of the plurality of A-scans using the sensor data. The processor may then construct the image in response to the plurality of positions of the measurement or imaging beam. In some embodiments, the position of the measurement or imaging beam is related to one or more of the position or the orientation of the measurement or imaging beam.

In some embodiments, the probe apparatus 501 may include the treatment channel 554 to treat the tissue. The movement sensor and the imager may be coupled to the probe 560, the imaging channel 552 and treatment channel 554 in a fixed relationship, such as a fixed position and orientation with respect to each other.

The one or more channels 550 may be shaped or otherwise configured to deliver one or more of an implant or a treatment energy to the tissue to treat the tissue, and combinations thereof. In some embodiments, the one or more channels 550 includes an optical fiber to deliver laser energy to the tissue. In some embodiments, the channel comprises a working channel to deliver the implant to the tissue.

As discussed herein, the probe apparatus 501 may include a housing 504. The housing may be a handheld housing, such as a handpiece which may be coupled to or include the imager and the sensor. In some embodiments, handheld devices are moved during use. Some movements are purposeful or intentional and some movements are unintentional. The movements sensor can measure both types of movements. One type of involuntary movement is tremor of the user, such as the surgeon, when holding the probe while treating a patient. This tremor movement may be relatively small, on the order of one or several millimeters or less. Because the structures of the eye a small, such small movements of the hand, even those that are less than a millimeter or less than 3 millimeters in amplitude, may be used to construct one or more of a 2D image, a 3D image or a 3D model or the treatment area of the eye. In some embodiments, a tremor model may be built to characterize the tremor of a user such as a surgeon. This tremor model, which may be based on the periodic movement, including the frequency and amplitude, of measured tremors of the user over time may be used to generate the sensor data and then further used with the imager data to generate 2D images, 3D images or a 3D model of the patient's tissue. In some embodiments, the processor may determine a position of the measurement or imaging beam in the tissue in based on the sensor data and the tremor model, for example.

Although reference is made to movements of the probe and structures such as the sensor, in some embodiments the tissue moves in relation to the probe, for example with repetitive movement as described herein. In some embodiments, the repetitive movement of the tissue is related to pulsatile movement as described herein. In some embodiments, the repeated movement, e.g. periodic movement, is measured with a detector as described herein. While the detector can be configured in many ways, in some embodiments the detector comprises an array detector. A processor can be configured to fit the movement data to periodic model, similar to a tremor or resonance model as described herein, and the movement data used to construct the 3D image, for example.

In some embodiments, an artificial intelligence model trained with imaging data of the eye may generate 2D images, 3D images or a 3D model of the patient's tissue from the A-scan data and one or more of a tremor model or a resonance model, for example. The position and orientation data can be used with the one or more of the tremor model or the resonance to develop the one or more of tremor model or the resonance model.

In some embodiments, the movement data comprises periodic movement related to pulsatile flow, such as movement of one or more tissues coupled to the cardiovascular system, such as one or more the Schlemm's canal, the collector channels, or the trabecular meshwork. Periodic data can be acquired and used to construct the 3D images as described herein. The periodic data may comprise any suitable periodic data related to one or more of tremor, resonance, or cardiac pulsation, for example. The movement data may comprise periodic movement data such as periodic movement related to one or more of tremor, resonance or pulsatile tissue movement. In some embodiments, the periodic movement corresponds to harmonics, which can be determined in response to the movement data. In some embodiments, the periodic movement corresponds to pulsations of tissue, such as the choroid under the retina, for example.

With reference to FIG. 5B an example probe apparatus 501 and imaging device 502 are illustrated in accordance with some embodiments. The imaging device 502 may comprise an ultrasound imaging device, for example. Portions of the imaging device 502 may be contained within a housing 504, such as a handheld housing, such as the control circuitry for an ultrasound transducer 565 configured to emit a measurement and receive beam with respect to axis 564. The ultrasound transducer 565 may be located near a tip 562 of the probe 560, and coupled to other portions of imaging device 502, for example. In some embodiments, one or more channels 550 may extend along a length of the probe 560. The treatment probe may be between 2 mm and 10 mm long, such as between 2.5 mm and 5 mm long.

In some embodiments, the one or more channels 550 comprises an imaging channel 552 and a treatment channel 554. In some embodiments, the imaging channel 552 comprises the ultrasound transducer 565 and associated wires to couple the transducer to the imaging device 502. In some embodiments, the treatment channel 554 comprises one or more optical fibers to deliver treatment light energy to the eye, or a channel to deliver an implant to the eye.

The transducer 565 of the ultrasound imaging device 502 may be configured in many ways and may comprise one or more of a single transducer or an array of transducers, for example. In The ultrasound imaging device 502 may image the eye with ultrasound A-scans, for example. During treatment, such as during imaging of the patient's eyes, the probe apparatus 501 may move in translation and rotation causing the ultrasound system 502 to generate A-scans from different positions and orientations of the measurement or imaging beam. The position and orientation data acquired with the sensor can be combined with the A-scans to generate an image of the tissue.

The probe apparatus 501 may include a movement sensor 520 that measures the movements of the probe apparatus 501 in three degrees of translation and three degrees of rotation. For example, the sensor 520 may measure movements in three degrees of translation and three degrees of rotation. The three degrees of translation may be three orthogonal axis, such as an x-axis 522, y-axis 524, and z-axis 526. The z-axis may be aligned with or parallel to the imaging and/or treatment axis 564 of the probe 560 while the x-axis and y-axis are in a plane substantially perpendicular to the z-axis, for example within about 10 degrees of perpendicular. The three degrees of rotation may be about the three orthogonal axes, such as a first degree of rotation 523 about the x-axis 522, a second degree of rotation 525 about the y-axis 525, and a third degree of rotation 527 about the z-axis.

The probe apparatus 501 may be coupled in electronic communication with a 3D imager 401 and a controlling unit 410, both of which are also described in further detail herein, such as with respect to FIG. 7. During operation, the imaging device 502, such as an ultrasound imaging device, may generate imaging data, such as A-scans while the sensor 520 measures the movement of the imaging device in both translation and rotation. The imaging data and movement data may be time-stamped or otherwise correlated with each other such that the position and orientation of the probe apparatus 501 may be associated with each A-scan of the ultrasound imaging device. The imaging data and the movement data may be processed by the 3D imager 401 and/or the controlling unit 410 in order to build a 3D model. The 3D model may be built as the ultrasound imaging device moves and captures data of different portions of the patient's eye. The 3D imager 401 and/or the controlling unit 410 may then assemble a 3D model by placing each A-scan or other ultrasound image data in spatial orientation with respect to other A-scans, based on the sensor movement data.

In some embodiments, the imaging device and the sensor may be coupled to a processor configured to acquire image data from the image sensor and sensor data, such as movement data, from the sensor and construct one or more of a 2D image, a 3D image or a 3D model based on or in response to the image data and the sensor data. The imaging device may have an axis, such as a z-axis along which it emits a beam of imaging energy, such as ultrasound. The axis of the beam may be aligned with respect to or parallel to at least one of axes 522, 524, 526 of the sensor for measuring one or more of the position or the orientation beam of imaging energy. The sensor may be configured to measure a movement of the imaging device and/or imaging beam in a direction along the axis of the beam corresponding to a movement of the measurement or imaging beam along the axis. The processor may then construct one or more of a 2D image, a 3D image or a 3D model in response to the movement of the measurement or imaging beam along the axis.

In some embodiments, the sensor is configured to measure a movement of the imager transverse to the axis 564 of the measurement or imaging beam. Such movement may be movement in a plane that is substantially perpendicular to the axis of the measurement or imaging beam (e.g. within about 10 degrees of perpendicular) and, in some embodiments, also substantially perpendicular to the z-axis (e.g. within about 10 degrees of perpendicular), such as a plane defined by the x-axis 522 and y-axis 524. The processor may then construct one or more of a 2D image, a 3D image or a 3D model in response to the movement of the measurement or imaging beam transverse to the axis.

In some embodiments, the movement data may include data of the rotation of the probe and attached imaging device. In some embodiments, the movement data may include data of the translation of the probe and attached imaging device.

In some embodiments, the processor or processors located in one or more of the 3D imager 401 and the controlling unit 410 may store data of the positions and orientations of the imager while the ultrasonic beam of imaging energy is directed toward the tissue and construct one or more of the 2D image, the 3D image or the 3D model from the plurality of positions and orientations of the sensor.

As discussed herein, the imager may generate A-scans with the ultrasonic measurement or imaging beam and the processor may determine one or more of a position or an orientation of the ultrasonic measurement or imaging beam for each of the plurality of A-scans using the sensor data. The processor may then construct the image in response to the plurality of positions of the ultrasonic measurement or imaging beam.

In some embodiments, the probe apparatus 501 may include the probe 560 and treatment channel 554 to treat the tissue. The movement sensor and the imager may be coupled to the probe 560, the imaging channel 552 and the treatment channel 554 in a fixed relationship, such as a fixed position and orientation with respect to each other.

The one or more channels 550 may be shaped or otherwise configured to deliver one or more of an implant of a treatment energy to the tissue to treat the tissue. In some embodiments, the one or more channels 550 includes an optical fiber to deliver laser energy to the tissue. In some embodiments, the channel comprises a working channel to deliver the implant to the tissue.

As discussed herein, the probe apparatus 501 may include a housing 504. The housing may be a handheld housing, such as a handpiece which may be coupled to or include the imager and the sensor. Handheld devices are moved during use. Some movements are purposeful or intentional and some movements are unintentional. The movements sensor can measure both types of movements. One type of involuntary movement is tremor data of the user, such as the surgeon, when holding the probe while treating a patient. This tremor movement may be relatively small, on the other of one or several millimeters or less. Because the structures of the eye a small, such small movements of the hand, even those that are less than a millimeter or less than 3 millimeters in amplitude, may be used to construct a 2D image, 3D image, or a 3D model or the treatment area of the eye. In some embodiments, a tremor model may be built to characterize the tremor of a surgeon. This tremor model, which may be based on the periodic movement, including the frequency and amplitude, of measured tremors of the surgeon over time may be used to generate the senor data and then further used with the imager data to generate 2D images, 3D images or a 3D model of the patient's tissue. In some embodiments, the processor may determine a position of the measurement or imaging beam in the tissue in based on the sensor data and the tremor model.

In some embodiments, an artificial intelligence model trained with imaging data of the eye may generate 2D images, 3D images or a 3D model of the patient's tissue from the A-scan data and a tremor model, such as without using position and orientation data.

With reference to FIG. 5C an example probe apparatus 501 and imaging device 502 are illustrated in accordance with some embodiments. The imaging device 502 may comprise a photoacoustic imaging device. The components of the imaging device 502 can be located at any suitable location, such as near the probe tip, within a housing of the handpiece, or within a console of a treatment station as described herein. Portions of the imaging device 502 may be contained within a housing 504, such as a handheld housing, such as the acoustic transducer circuitry portions 568 of the photoacoustic imaging device. The sensor may be located on an external surface of the housing 504 or be acoustically coupled to the external environment outside the housing. In some embodiments, the sensor comprises an ultrasound detector for detecting sound waves created when light from imaging device contacts the tissue. In some embodiments, the acoustic transducer 565 may be located on a distal end of the probe, such as the tip. In some embodiments, one or more channels 550 may extend along a length of the probe 560. The treatment channel of the probe 560 may be between 2 mm and 10 mm long, such as between 2.5 mm and 5 mm long. The imaging device 502, including the imaging sensor may be between 2 mm and 10 mm from the tissue, such as between 2.5 mm and 5 mm from the tissue of the eye during use. The one or more channels 550 may be a light guiding channel that guides light from the photoacoustic transducer out the end of the treatment probe to the tissue. The one or more channels 550 may include an optical fiber for transmitting light out the tip 562 of the probe.

In some embodiments, the one or more channels 550 comprises an imaging channel 552 and a treatment channel 554. In some embodiments, the imaging channel 552 comprises an optical fiber to transmit the photoacoustic excitation beam to induce vibrations in the tissue that can be measured with the transducer 567. In some embodiments, the treatment channel 554 comprises one or more optical fibers to deliver treatment light energy to the eye, or a channel to deliver an implant to the eye. Although reference is made to separate imaging and treatment channels, in some embodiments the two channels are combined into a single channel, for example when a single optical fiber is used for the photoacoustic excitation beam and the laser treatment beam in a multiplexed configuration.

The distal end of the probe 560 may be formed with an inclined surface having an angle relative to the longitudinal axis of the probe 560, for example to contact tissue of the trabecular meshwork with a beveled end of an optical fiber extending along treatment channel 554.

In some embodiments, a single optical fiber may be used for the treatment channel 554 and imaging channel 554. In some embodiments, a bundle of optical fibers, such as two or more fibers could be used with the disclosed systems and methods. In some examples, the probe 560 comprises a bundle of optical fibers that each have a distal end at or near the angle of the distal end of the treatment probe 500.

The photoacoustic imaging device 502 may image the eye with photoacoustic A-scans. During treatment, such as during imaging of the patient's eyes, the probe apparatus 501 may move in translation and rotation causing the photoacoustic system 502 to generate A-scans from different positions and orientations.

The probe apparatus 501 may include a movement sensor 520 as described herein, and the movement data can be measured and combined with image data as described herein.

The probe apparatus 501 may be coupled in electronic communication with a 3D imager 401 and a controlling unit 410, both of which are also described in further detail herein, such as with respect to FIG. 7. During operation, the imaging device 502, such as a photoacoustic imaging device, may generate imaging data, such as A-scans while the sensor 520 measures the movement of the imaging device in both translation and rotation. The imaging data and movement data may be time-stamped or otherwise correlated with each other such that the position and orientation of the probe apparatus 501 may be associated with each A-scan of the photoacoustic imager. The imaging data and the movement data may be processed by the 3D imager 401 and/or the controlling unit 410 in order to build a 3D model. The 3D model may be built as the photoacoustic imaging device moves and captures data of different portions of the patient's eye. The 3D imager 401 and/or the controlling unit 410 may then assemble a 3D model by placing each A-scan in spatial orientation with respect to each other A-scan, based on the sensor movement data.

In some embodiments, the imaging device and the sensor may be coupled to a processor configured to acquire image data from the image sensor and sensor data, such as movement data, from the sensor and construct one or more of a 2D image, a 3D image or a 3D model based on or in response to the image data and the sensor data as described herein.

In some embodiments, the sensor is configured to measure a movement of the imager transverse to the axis 564 of the measurement or imaging beam. Such movement may be movement in a plane that is substantially perpendicular to the axis of the measurement or imaging beam (e.g. within 10 degrees of perpendicular) and, in some embodiments, also substantially perpendicular to the z-axis (e.g. within 10 degrees of perpendicular), such as a plane defined by the x-axis 522 and y-axis 524. The processor may then construct one or more of a 2D image, 3D image, or a 3D model in response to the movement of the measurement or imaging beam transverse to the axis.

In some embodiments, the movement data may include data of the rotation of the probe and attached imaging device. In some embodiments, the movement data may include data of the translation of the probe and attached imaging device.

In some embodiments, the processor or processors located in one or more of the 3D imager 401 and the controlling unit 410 may store data of the positions and orientations of the imager while the beam of imaging energy is directed toward the tissue and construct the 2D images, 3D images or a 3D model from the plurality of positions and orientations of the sensor.

As discussed herein, the imager may generate A-scans with the measurement or imaging beam and the processor may determine a position of the measurement or imaging beam for each of the plurality of A-scans using the sensor data. The processor may then construct the image in response to the plurality of positions of the measurement or imaging beam.

In some embodiments, the probe apparatus 501 may include the treatment channel 554 to treat the tissue. The movement sensor and the imager may be coupled to the probe 560, the imaging channel 552 and the treatment channel 554 in a fixed relationship, such as a fixed position and orientation with respect to each other.

The treatment channel 554 of the one or more channels 550 may be shaped or otherwise configured to deliver one or more of an implant or a treatment energy to the tissue to treat the tissue. In some embodiments, the one or more channels 550 includes an optical fiber to deliver laser energy to the tissue. In some embodiments, the channel comprises a working channel to deliver the implant to the tissue.

As discussed herein, the probe apparatus 501 may include a housing 504. The housing may be a handheld housing, such as a handpiece which may be coupled to or include the imager and the sensor. Handheld devices are moved during use. Some movements may be purposeful or intentional and some movements unintentional. The movements sensor can measure both types of movements. One type of involuntary movement is tremor data of the user, such as the surgeon, when holding the probe while treating a patient. This tremor movement may be relatively small, on the other of one or several millimeters or less. Because the structures of the eye a small, such small movements of the hand, even those that are less than a millimeter or less than 3 millimeters in amplitude, may be used to construct a 2D image, 3D image, or a 3D model or the treatment area of the eye. In some embodiments, a tremor model may be built to characterize the tremor of a surgeon. This tremor model, which may be based on the periodic movement, including the frequency and amplitude, of measured tremors of the surgeon over time may be used to generate the senor data and then further used with the imager data to generate 2D images, 3D images or a 3D model of the patient's tissue. In some embodiments, the processor may determine a position of the measurement or imaging beam in the tissue in based on the sensor data and the tremor model.

In some embodiments, an artificial intelligence model trained with imaging data of the eye may generate 2D images, 3D images or a 3D model of the patient's tissue from the A-scan data and a tremor model as described herein.

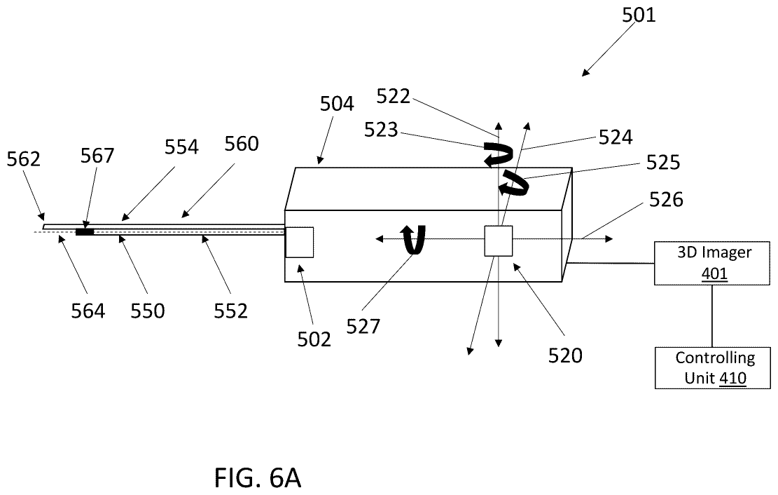

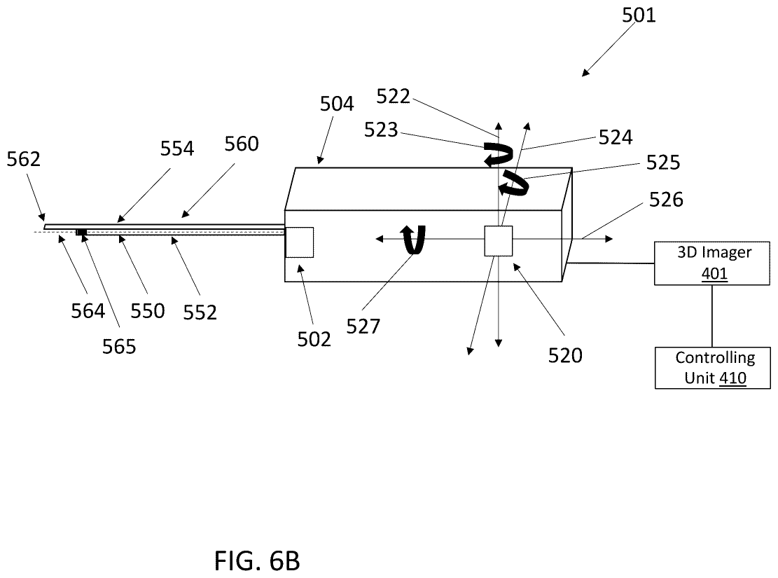

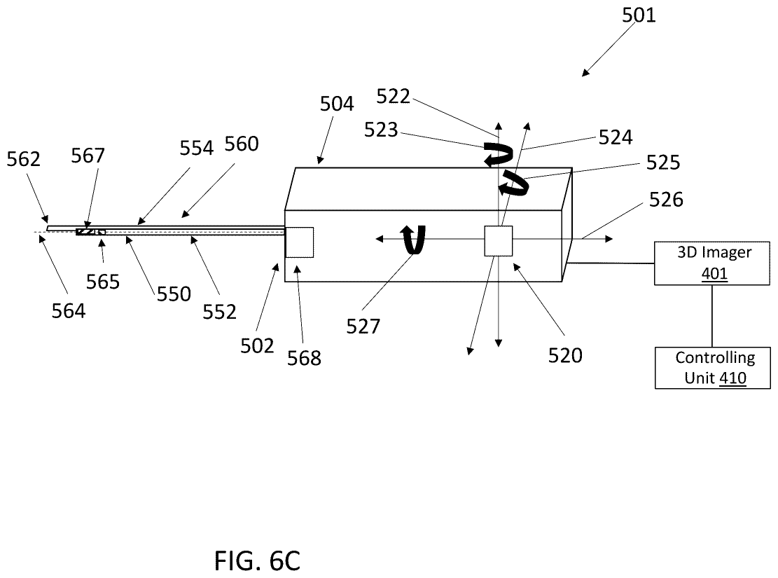

With reference to FIG. 6A an example probe apparatus 501 and imaging device 502 are illustrated in accordance with some embodiments. Which may be similar to and have similar features as the probe apparatus 501 of FIGS. 5A to 5C. The imaging device 502 may comprise a scanning OCT imaging device. Portions of the imaging device 502 may be contained within a housing 504, such as a handheld housing, such as the interferometer portions of the scanning OCT imaging device, including the OCT light source, beam splitter, and reference and sample a detector array(s). In some embodiments, one or more channels 550 may extend along a length of the probe 560. The treatment channel 554 of the probe may be between 2 mm and 10 mm long, such as between 2.5 mm and 5 mm long, for example. The one or more channels 550 may be a light guiding channel that guides light from the OCT system out the end of the treatment probe. The one or more channels 550 may include an optical fiber for transmitting light out the tip 562 of the probe.

In some embodiments, the one or more channels 550 comprises an imaging channel 552 and a treatment channel 554. In some embodiments, the imaging channel 552 comprises an optical fiber to transmit and receive the OCT measurement or imaging beam. In some embodiments, the treatment channel 554 comprises one or more optical fibers to deliver treatment light energy to the eye, or a channel to deliver an implant to the eye. In some embodiments, the distal tip of the imaging channel 552 is located proximally in relation to the distal tip of the treatment channel in order to image tissue and the distal tip of the treatment channel as described herein. Although reference is made to separate imaging and treatment channels, in some embodiments the two channels are combined into a single channel, for example when a single optical fiber is used for the OCT measurement or imaging beam and the laser treatment beam in a multiplexed configuration.

The OCT imaging device may comprise any suitable OCT imaging device, such as a broad spectrum imaging device with a movable mirror, a Fourier domain imaging device, a spectral domain OCT imaging device, or a swept source OCT imaging device, for example, as will be understood by one of ordinary skill in the art.

The distal end of the probe 560 may include a scanner 567 that scans the OCT measurement or imaging beam in a pattern with respect to a measurement axis, such as axis 564, such as by deflecting the distal end of the OCT imaging fiber or measurement or imaging beam, such as with piezo electric deflection, piezo electric deflection of an optical fiber, galvanic deflection or moving reflective surface, such as a mirror. In some embodiments, the scanner comprises a single OCT imaging fiber configured to deflect to scan the imaging beam. Scanning optical fibers suitable for incorporation in accordance with the present disclosure are described in PCT/US2001/016844, filed May 23, 2001, entitled “Medical imaging, diagnosis, and therapy using a scanning single optical fiber system”, published as WO2001097902 on Dec. 27, 2001.

Although reference is made to scanners, in some embodiments, the processor is configured to combine tremor data with data from the scanning imaging beam to construct the 3D image as described herein.

The scanning OCT imaging device 502 may image the eye with OCT A-scans scanned over the tissue of the patient using the scanner 562. During treatment, such as during imaging of the patient's eyes, the probe apparatus 501 may move in translation and rotation causing the OCT system 502 to generate a plurality of scanning A-scans from scanner 567 with different positions and orientations of the probe that are measured with sensor 520.

The probe apparatus 501 may include a movement sensor 520 that measures the movements of the probe apparatus 501 in three degrees of translation and three degrees of rotation as discussed herein.

The probe apparatus 501 may be coupled in electronic communication with a 3D imager 401 and a controlling unit 410, both of which are also described in further detail herein, such as with respect to FIG. 7. During operation, the imaging device 502, such as an OCT imaging device, may generate imaging data, such as A-scans in the scan pattern while the sensor 520 measures the movement of the imaging device in both translation and rotation. The imaging data, including the position of the measurement or imaging beam within a scan pattern, and movement data may be time-stamped or otherwise correlated with each other such that the position and orientation of the probe apparatus 501 may be associated with each A-scan of the OCT scanner. The imaging data and the movement data may be processed by the 3D imager 401 and/or the controlling unit 410 in order to build a 3D model. The 3D model may be built based on data acquired as the OCT imaging device moves and captures data of different portions of the patient's eye. The 3D imager 401 and/or the controlling unit 410 may then assemble a 3D model by placing each A-scan in spatial orientation with respect to each other A-scan, based on the sensor movement data, as discussed herein, such as with respect to FIG. 5A, including using tremors for probe movement.

With reference to FIG. 6B an example probe apparatus 501 and imaging device 502 are illustrated in accordance with some embodiments. Which may be similar to and have similar features as the probe apparatus 501 of FIG. 5B. The imaging device 502 may comprise an ultrasound imaging device. Portions of the imaging device 502 may be contained within a housing 504, such as a handheld housing, such as the control circuitry for an ultrasound transducer 565. The ultrasound transducer 565 may be located near a tip 562 of the probe 560. The ultrasound transducer 565 may include an array of sensors, such as a 1-D, linear array or a 2-D area or planar array. The treatment probe may be between 2 mm and 10 mm long, such as between 2.5 mm and 5 mm long.

In some embodiments, the one or more channels 550 comprises an imaging channel 552 and a treatment channel 554. In some embodiments, the imaging channel 552 comprises the ultrasound transducer 565 and associated wires to couple the transducer to the imaging device 502. In some embodiments, the treatment channel 554 comprises one or more optical fibers to deliver treatment light energy to the eye, or a channel to deliver an implant to the eye.

The ultrasound system 502 may image the eye with ultrasound A-scans or with a beam former generating 2D or 3D images, for example. During treatment, such as during imaging of the patient's eyes, the probe apparatus 501 may move in translation and rotation causing the ultrasound system 502 to generate imaging scans from different positions and orientations.

The probe apparatus 501 may include a movement sensor 520 that measures the movements of the probe apparatus 501 in three degrees of translation and three degrees of rotation. For example, the sensor 520, which may be referred to as a movement sensor, may measure movements in three degrees of translation and three degrees of rotation, as discussed herein, such as with respect to FIG. 5B.

The probe apparatus 501 may be coupled in electronic communication with a 3D imager 401 and a controlling unit 410, both of which are also described in further detail herein, such as with respect to FIG. 7. During operation, the imaging device 502, such as an ultrasound imaging device, may generate imaging data, such as A-scans formed by the 1D or 2D sensor array while the sensor 520 measures the movement of the imaging device in both translation and rotation. The imaging data and movement data may be time-stamped or otherwise correlated with each other such that the position and orientation of the probe apparatus 501 may be associated with each A-scan of the ultrasound imaging device. The imaging data and the movement data may be processed by the 3D imager 401 and/or the controlling unit 410 in order to build a 3D model. The 3D model may be built because, as the ultrasound imaging device moves it captures data of different portions of the patient's eye. The 3D imager 401 and/or the controlling unit 410 may then assemble a 3D model by placing each A-scan in spatial orientation with respect to each other A-scan, based on the sensor movement data, as discussed herein, such as with respect to FIG. 5B, including using tremors or resonance modes of a robotic arm for probe movement.

In some embodiments, the imaging device and the sensor may be coupled to a processor configured to acquire image data from the image sensor and sensor data, such as movement data, from the sensor and construct one or more of a 2D image, a 3D image or a 3D model based on or in response to the image data and the sensor data. The imaging device may have an axis, such as a z-axis along which it emits a beam of imaging energy, such as ultrasound. The axis of the beam may be aligned with respect to or parallel to at least one of axes 522, 524, 526 of the sensor for measuring one or more of the position or the orientation beam of imaging energy. In some embodiments, the sensor array may be oriented substantially perpendicular to the axis 564, for example to within 10 degrees of perpendicular. For example, a 1-D array may be substantially perpendicular and the plane of a 2D array may be substantially perpendicular to the axis 564. The sensor may be configured to measure a movement of the imaging device and/or imaging beam in a direction along the axis of the beam corresponding to a movement of the measurement or imaging beam along the axis. The processor may then construct one or more of a 2D image, a 3D image or a 3D model in response to the movement of the measurement or imaging beam along the axis.

In some embodiments, the sensor is configured to measure a movement of the imager transverse to the axis 564 of the measurement or imaging beam. Such movement may be movement in a plane that is substantially perpendicular to the axis of the measurement or imaging beam (e.g. to within 10 degrees of perpendicular) and, in some embodiments, also substantially perpendicular (e.g. to within 10 degrees of perpendicular) to the z-axis, such as a plane defined by the x-axis 522 and y-axis 524. The processor may then construct one or more of a 2D image, a 3D image or a 3D model in response to the movement of the measurement or imaging beam transverse to the axis.

With reference to FIG. 6C an example probe apparatus 501 and imaging device 502 are illustrated in accordance with some embodiments. Which may be similar to and have similar features as the probe apparatus 501 of FIG. 5C. The imaging device 502 may comprise a photoacoustic imaging device. Portions of the imaging device 502 may be contained within a housing 504, such as a handheld housing, such as the transducer portions 568 of the photoacoustic imaging device. In some embodiments, the sensor 565 comprises an ultrasound detector for detecting sound waves created when light from imaging device contacts the tissue and induces vibrations. In some embodiments, the acoustic sensor may be located on a distal end of the probe, such as the tip. In some embodiments, one or more channels 550 may extend along a length of the probe 560. The treatment probe may be between 2 mm and 10 mm long, such as between 2.5 mm and 5 mm long. The imaging device 502, including the imaging sensor may be between 2 mm and 10 mm from the tissue, such as between 2.5 mm and 5 mm from the tissue of the eye during use. The one or more channels 550 may comprise a light guiding channel that guides light from the photoacoustic transducer out the end of the treatment probe. The one or more channels 550 may include an optical fiber for transmitting light out the tip 562 of the probe.

In some embodiments, the one or more channels 550 comprises an imaging channel 552 and a treatment channel 554. In some embodiments, the imaging channel 552 comprises an optical fiber to transmit the photoacoustic excitation beam to induce vibrations in the tissue that can be measured with the transducer 567. In some embodiments, the treatment channel 554 comprises one or more optical fibers to deliver treatment light energy to the eye, or a channel to deliver an implant to the eye. Although reference is made to separate imaging and treatment channels, in some embodiments the two channels are combined into a single channel, for example when a single optical fiber is used for the photoacoustic excitation beam and the laser treatment beam in a multiplexed configuration.

The distal end of the probe 560 may include a scanner 567 that scans the measurement or imaging beam in a pattern with respect to a measurement axis, such as axis 564, such as by deflecting the distal end of the photoacoustic beam, such as with one or more of piezo electric deflection, optical fiber deflection, piezo electric deflection of an optical fiber, galvanic deflection or moving reflective surface, such as a mirror.

The photoacoustic device 502 may image the eye with photoacoustic scans scanned over the tissue of the patient using the scanner 567. During treatment, such as during imaging of the patient's eyes, the probe apparatus 501 may move in translation and rotation causing the photoacoustic system 502 to generate A-scans from different positions and orientations.

The probe apparatus 501 may include a movement sensor 520 that measures the movements of the probe apparatus 501 in three degrees of translation and three degrees of rotation. For example, the sensor 520, which may be referred to as a movement sensor, may measure movements in three degrees of translation and three degrees of rotation, as discussed herein.

The probe apparatus 501 may be coupled in electronic communication with a 3D imager 401 and a controlling unit 410, both of which are also described in further detail herein, such as with respect to FIG. 7. During operation, the imaging device 502, such as a photoacoustic imaging device, may generate imaging data, such as A-scans in the scan pattern while the sensor 520 measures the movement of the imaging device in both translation and rotation. The imaging data, including the position of the beam within a scan pattern, and movement data may be time-stamped or otherwise correlated with each other such that the position and orientation of the probe apparatus 501 may be associated with each A-scan of the photoacoustic imager. The imaging data and the movement data may be processed by the 3D imager 401 and/or the controlling unit 410 in order to build a 3D model. The 3D model may be built because, as the photoacoustic imaging device moves it captures data of different portions of the patient's eye. The 3D imager 401 and/or the controlling unit 410 may then assemble a 3D model by placing each A-scan in spatial orientation with respect to each other A-scan, based on the sensor movement data, as discussed herein, such as with respect to FIG. 5C, including using tremors for probe movement.

Although FIGS. 5A to 6C refer to a combined imaging and treatment probe, in some embodiments the probe comprises an imaging probe without a treatment channel. Alternatively, the probe can be configured for treatment without imaging. In some embodiments, the components of the probes shown in FIGS. 5A to 6C are provided as a plurality of probes that can be used together for treatment, for example with a first treatment probe to treat tissue and a second imaging probe to image the tissue. In some embodiments, the imaging probe and the treatment probe are inserted through different incisions. For example, the imaging probe can be inserted through a first incision and the treatment probe inserted through a second incision, and the imaging probe used to image tissue and the treatment probe simultaneously.

With reference to FIG. 7, a system 400 for aiding a surgeon to perform a surgical procedure on an eye E, is illustrated in accordance with some embodiments. The surgical operation procedure may comprise inserting a portion of an elongate probe apparatus 501 from an opening into the eye across an anterior chamber to a target tissue region comprising a trabecular meshwork and a Schlemm's canal. In some embodiments, the system 400 may comprise an optical microscope 409 for the surgeon to view the eye during the procedure in real-time. A 3D imager 401 receives a feed from the imaging device of the elongate probe apparatus 501, which may include a portion of the systems described with reference to FIGS. 5A to 6C, placed in or proximate the eye as input. The 3D imager 401 is operatively coupled a processor 414 of the controlling unit 410. The processor of the controlling unit 410 can be configured with instructions to identify locations of structures of the eye and overlay indicia such as markers on the input camera images. In conjunction with the optical microscope 409, an imaging device coupled to probe apparatus 501 as described herein may provide a data to 3D imager 401 and the controlling unit 410. In some embodiments, a second camera 416 comprising a detector array is optically coupled to the microscope 409 to receive optical images from the operating microscope 409, and optically coupled to the processor 414 of the control unit 410. The control unit 410 can receive the image data from the camera 416 and process the image data to provide visual image data on the heads-up display 407 and overlay the visual image data on an anterior optical image of the operating microscope 409. The microscope 409 may comprise a binocular surgical operating microscope, for example. The system 400 may comprise an imaging device of probe apparatus 501, or a portion thereof, that is delivered in situ along with the treatment probe 23 or immediately proximate the location of the treatment probe 23 and the eye to provide imaging of one or more target locations before, during, or after the procedure, for example. The imaging device of the probe apparatus 501, may comprise one or more components as described with reference to FIGS. 5A to 6C, here, including OCT imaging, ultrasound imaging, and photoacoustic imaging and the sensor to measure one or more of the position or orientation of the prob as described herein. Images captured by the imaging device may be processed by an image processing apparatus 412 of the controlling unit 410 to generate a plurality of 2D and 3D images or models and augmented images and models for visualizations by the surgeon in real time.

The augmented images can be shown on a display of the heads up display 407, and combined with optical images from the microscope 409 with an internal beam splitter 420 to form monocular or binocular images as is known to one of ordinary skill in the art. As described herein, a microscope view may comprise one or more of an optical microscope image, a microscope image and an overlaid virtual image, or a microscope image in combination with one or more of 2D images, 3D images or models generated from imaging captured by the imaging device with or without an overlaid virtual image, for example. When a microscope view includes an overlaid image, the overlaid image can be registered with the microscope image using elements which enable such alignment. Similarly, when the view includes imaging from the camera and an overlaid virtual image, the overlaid image can be registered with the imaging from the camera using elements which enable such alignment.

The images can be provided to the surgeon in many ways. For example, the surgeon can view the images with an augmented reality display such as glasses or goggles and view the surgical site through the operating microscope 409. In some embodiments, the surgeon views the images with a virtual reality display. Alternatively or in combination, the eye can be viewed with an external monitor, and the images of the eye viewed with the external monitor with markings placed thereon as described herein. The images viewed by the surgeon may comprise monocular images or stereoscopic images, for example. In some embodiments, the images viewed by the surgeon comprise augmented reality (AR) images or virtual reality images, for example.

According to some embodiments, a surgeon may first view a surgical instrument, such as a portion of probe apparatus 501, in the microscope or a video image from the operating microscope. In some cases, the surgeon may alternatively, or additionally, view images, such as 2D or 3D images or models generated from data by the imaging device of the probe apparatus 501. According to some embodiments, a surgeon may view images from the microscope 409 and 2D or 3D images or models generated from data by the imaging device of the probe through the oculars of the microscope 409. Alternatively or in combination, the surgeon may view an augmented image or view, where additional information is overlaid on one or more of the optical microscope image or the 2D or 3D images or models generated from data by the imaging device. When there is a 2D or 3D images or models generated from data by the imaging device overlaid on the image from the microscope image, the surgeon can view both the microscope image and concurrently the overlaid 2D or 3D images or models generated from data by the imaging device. Furthermore, the image processing apparatus 412 can detect anatomical features of the eye as described herein and overlay markers onto the microscope image or one or more of the 2D images, the 3D images or models generated from data by the imaging device to help guide a surgeon in identifying and locating these features. The augmented images may be presented to the surgeon through an eyepiece (or eyepieces) or oculars of the microscope and/or a display of the microscope, and in some embodiments may be viewed on a monitor screen. This may be beneficial to allow a surgeon to maintain a stereoscopic view of an operative site through the oculars of the microscope while simultaneously viewing superimposed or adjacent images or information concurrently either stereoscopically or monocularly, for example. Real-time 2D or 3D images or models generated from data by the imaging device of probe apparatus 501 in situ and real time treatment information can be superimposed to the live view of one or both oculars. In some embodiments, the apparatus and methods disclosed provide a real-time view including real and augmented images from both outside and inside of the anterior chamber during these surgeries.

The optical microscope 409 may be operatively coupled to the imaging device of probe apparatus 501 when inserted into the eye in many ways. The optical microscope 409 may comprise a binocular microscope such as a stereomicroscope comprising imaging lens elements to image an object onto an eyepiece(s) comprising an ocular 408. The imaging device of probe apparatus 501 placed in, on, or about the eye is configured to capture images of the eye and may comprise any of the imaging systems described herein, such as those described with respect to FIGS. 5A to 6C. The optical images may be transmitted to the controlling unit 410 for processing.

The imaging device of probe apparatus 501 may comprise emitters and sensors as described herein. Parts of the imaging device may be introduced with the treatment probe apparatus 501 and moved with the treatment probe apparatus 501, or the treatment probe apparatus 501 may move independently of the imaging device while maintaining alignment with the treatment probe apparatus 501. The probe apparatus 501 may be configured with a handpiece as described herein to allow insertion, manipulation, or withdrawal of the probe apparatus 501, such as by a user, an actuator, a robotic arm, or otherwise.

In some embodiments, the optical microscope 409 may be coupled to an electronic display device 407. The electronic display 407 may comprise a heads-up display device (HUD). The HUD may or may not be a component of the microscope system 409. The HUD may be optically coupled into the field-of-view (POV) of one or both of the oculars 408. The display device may be configured to project augmented images from input 401 generated by the controlling unit 410 to a user or surgeon. The display device 407 may alternatively or additionally be configured to project images captured by the camera and/or imaging device to a user or surgeon. The display device may be coupled to the microscope via one or more optical elements such as beam-splitter or mirror 420 such that a surgeon looking into the eyepieces 408 can perceive in addition to the real image, camera imaging, augmented images, one or more of 2D images, 3D images or models generated using data from the imaging device, or any combination represented and presented by the display device 407. The display device may be visible through a single ocular to the surgeon or user. Alternatively, the HUD may be visible through both eyepieces 408 and visible to the surgeon as a stereoscopic binocular image combined with the optical image formed with components of the microscope, for example.

In some embodiments, the display device of heads-up display 407 is in communication with the controlling unit 410. The display device may provide augmented images produced by the controlling unit 410 in real-time to a user. As described herein, real time imaging may comprise capturing the images or image data and generating 2D or 3D images or models with no substantial latency and allows a surgeon the perception of smooth motion flow that is consistent with the surgeon's tactile movement of the surgical instruments during surgery. In some cases, the display device 407 may receive one or more control signals from the controlling unit 410 for adjusting one or more parameters of the display such as brightness, magnification, alignment and the like. The image viewed by a surgeon or user through the oculars or eyepieces 408 may be a direct optical view of the eye, images displayed on the display 407 or a combination of both. Therefore, adjusting a brightness of the images on the HUD may affect the view of the surgeon through the oculars. For instance, processed information and markers shown on the display 407 can be balanced with the microscope view of the object. The processor may process the camera image data, such as to increase contrast of the image data so the visible features are more readily detectable or identifiable.

The heads up display 407 may be, for example, a liquid crystal display (LCD), a LED display, an organic light emitting diode (OLED), a scanning laser display, a CRT, or the like as is known to one of ordinary skill in the art.

Alternatively or in combination, the display 407 may comprise an external display. For example, the display 407 may not be perceivable through the oculars in some embodiments. The display 407 may comprise a monitor located in proximity to the optical microscope 409. The display 407 may comprise a display screen, for example. The display 407 may comprise a light-emitting diode (LED) screen, OLED screen, liquid crystal display (LCD) screen, plasma screen, or any other type of screen. The display device 407 may or may not comprise a touchscreen. A surgeon may view real-time optical images of the surgical site and imaging provided by the imaging device 702 simultaneously from the display 407.

The resolution of the imaging device of the probe apparatus 501 can be configured in many ways with appropriate optics and/or sensor resolution to image the target tissue at an appropriate resolution. The imaging systems may generate 2D and 3D images and models with a suitable resolution for viewing tissue structures of the eye as described herein and may comprise a resolution within a range from 1 to 10 microns, for example within a range from about 3 to 6 microns, for example. In some embodiments, the imaging systems may generate 2D and 3D images and models, comprises a spatial resolution, e.g. image spatial resolution, within a range from about 10 μm to about 80 μm for tissue contacting the inclined distal end of the probe (or contacting the implant). In some embodiments, the resolution is within a range from about 20 μm to about 40 μm.

The system 400 may further comprise a user interface 413. The user interface 413 may be configured to receive user input and provide output information to a user. The user input may be related to control of a surgical tool such as the probe apparatus 501. The user interface 413 may receive an input command related to the operation of the optical microscope (e.g., microscope settings, camera acquisition, etc.). The user interface 413 may receive an Indication related to various operations or settings about the camera. For instance, the user input may include a selection of a target location, a selection of a treatment reference marker, displaying settings of an augmented image, customizable display preferences and the like. The user interface 413 may include a screen such as a touch screen and any other user interactive external device such as handheld controller, mouse, joystick, keyboard, trackball, touchpad, button, verbal commands, gesture-recognition, attitude sensor, thermal sensor, touch-capacitive sensors, foot switch, or any other device.

The controlling unit 410 may be configured to generate an augmented layer comprising the augmented information. The augmented layer may be a substantially transparent image layer comprising one or more graphical elements. The terms “graphical element” and “graphical visual element” may be used interchangeably throughout this application. The augmented layer may be superposed onto the optical view of the microscope, optical images or video stream, and/or displayed on the display device. In some embodiments, the augmented layer is superimposed onto the optical view of the microscope, such that the transparency of the augmented layer allows the optical image to be viewed by a user with graphical elements overlaid on top of it. In some embodiments, the augmented layer may comprise real time images such as one or more of real time 2D image, 3D images or models as described herein obtained by one or more of the imaging devices of probe apparatus 501 placed in, on, or proximate the eye.

In some embodiments, the graphical elements may be configured to dynamically change as a position or an orientation of the probe or instrument changes relative to a target location. For example, a graphical element may indicate a location of a distal end of the probe shown in the optical image, or relative location or spacing between tissues such as inner wall of SC, TM and the like. The graphical elements may be configured to dynamically show the change in spacing between the tissue walls or distance between the tip and a target location substantially in or near real-time on the optical image, as the relative distance between the probe tip and a target location changes, and/or when the probe tip compresses on tissue (e.g., the probe tip contacting the surface of trabecular meshwork).

The augmented layer or at least some of the graphical elements can be mapped or matched to the optical image using object recognition techniques or pattern matching techniques, such as feature point recognition, edge detection, classifiers, spatial pyramid pooling, convolutional neural networks, or any of a number of suitable object recognition algorithms, or a combination of techniques. A feature point can be a portion of an image (e.g., scleral landmarks, collector channel patterns, iris landmarks, etc.) that is uniquely distinguishable from the remaining portions of the image and/or other feature points in the image. A feature point may be detected in portions of an image that are relatively stable under perturbations (e.g., when varying illumination and brightness of an image).

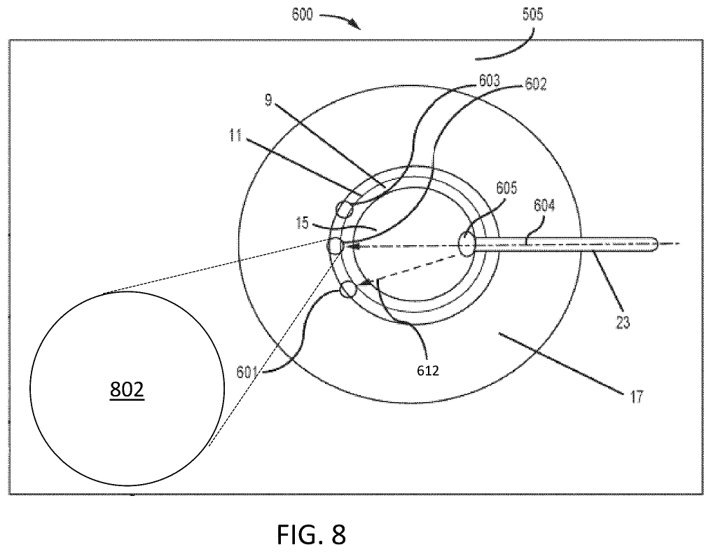

With reference to FIG. 8, an exemplary augmented image providing an augmented view 600 is shown. As described herein, the augmented image 600 may be viewed binocularly by a user or surgeon through oculars of the microscope, and may be displayed on a heads-up display, an external display device, or a display coupled to a user interface. The augmented image or view may comprise an optical image 505 or an optical path view through the oculars of an optical microscope. The optical image 505 may comprise a top-down view of the eye. The optical image 505 or optical view may show anterior portion of the eye. The optical image 505 or optical view may further show a portion elongate probe apparatus 501. The augmented image or view 600 may comprise a plurality of graphical visual elements and/or one or more of the 2D images, 3D images or models 802 from the probe apparatus 501 as described herein overlaid over the optical image, for example by optically coupling the display to the optical path of the microscope, such as with a beam splitter. The plurality of graphical visual elements may comprise different shapes and/or colors corresponding to different objects such that different objects shown in the optical image can be easily distinguished from one another. For example, one or more of the 2D images, the 3D images or the models may be overlaid with an identification and location of Schlemm's Canal, such as a Schlemm's canal identifier. In some embodiments, one or more of the 2D images, 3D images or models 802 from the probe apparatus 501 comprise one or more of OCT images, ultrasound images, or photoacoustic images as described herein overlaid on optical image 505.

The plurality of graphical visual elements may comprise one or more treatment reference markers 601, 602, 603 mapped to the one or more target locations. As discussed elsewhere herein, treatment reference markers 601, 602, 603 may correspond to target locations which are not optically visible to the surgeon in the optical image from the operating microscope. According to some embodiments, target locations may be located ab interno, and treatment of the target locations may involve an ab interno approach. Alternatively or in combination, the target locations may be located ab externo, for example with a femto second laser configured to deliver laser energy through one or more of the sclera or the cornea. Examples of laser delivery systems and femto second lasers suitable for incorporation in accordance with the present disclosure are described in U.S. application Ser. No. 14/732,627, filed on Jun. 5, 2015, “entitled Methods and apparatuses for the treatment of glaucoma using visible and infrared ultrashort laser pulses”, published as US20160095751 on Apr. 7, 2016, the entire disclosure of which is incorporated herein by reference.

The plurality of graphical visual elements may also comprise a probe line 604 coaxial with the elongate probe. The probe line 604 shows an orientation of the probe in relation to the one or more target locations. The plurality of graphical visual elements may also comprise a distal tip marker 605 overlapping with the distal end of the elongated probe. Both of the probe line and the distal tip marker may dynamically change locations with respect to the actual positions and orientation of the elongate probe shown in the optical image or view 802, as the probe is moved within the anterior chamber of the eye. Hence, for example, a surgeon can use microscope to see the probe as it enters the anterior chamber and can watch the probe as it moves relative to the eye. A detection mechanism can detect the probe, and an automated system or processor can generate the probe line 604 in response to the detection. Similarly, the automated system or processor can generate the guidance arrow 612.

The plurality of graphical visual elements may further comprise one or more guidance arrows or markers 612 extending from the distal tip marker 605 towards the one or more treatment reference markers (e.g., marker 601). The one or more guidance arrows 612 may be configured to guide the surgeon in aligning the distal end of the elongate probe to point towards the one or more target locations during the procedure or guide the surgeon in advancing the elongate probe towards the one or more target locations during the procedure. As discussed elsewhere herein, the one or more target locations may not be optically visible to the surgeon in the microscope view 505, and the camera imaging may be superimposed to allow the surgeon to see real-time imaging of the distal tip of the probe.

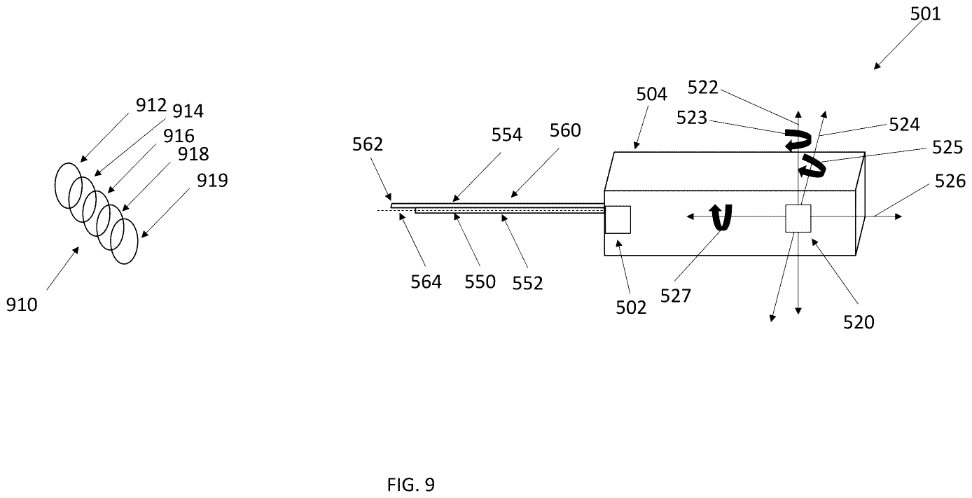

FIG. 9 shows movement of probe 560 and corresponding positions 910 of a measurement or imaging beam. In some embodiments, movement of the probe to a plurality of positions and orientations, such as may result from tremor, results in the measurement or imaging beam moving to a plurality of positions and orientation corresponding to the position and orientation of the probe 560 and probe apparatus 501. The plurality of positions may comprise a first position 912 corresponding to a first position and orientation of probe 560 and probe apparatus 501, a second position 914 corresponding to a second position and orientation of probe 560 and probe apparatus 501, a third position 916 corresponding to a third position and orientation of probe 560 and probe apparatus 501, a fourth position 918 corresponding to a fourth position and orientation of probe 560 and probe apparatus 501, and a fifth position 918 corresponding to a fifth position and orientation of probe 560 and probe apparatus 501. In some embodiments, each of the plurality of positions corresponds to a measurement location of the beam during a scan such as an A-scan and the position and orientation of the probe measured for each of the plurality of A-scans. The position and orientation data may comprise position and orientation data measured for each of the first axis 522, the second axis 524, and the third axis 526, for example.

In some embodiments, a plurality of position and orientation measurements are fit to a model. Work in relation to the present disclosure suggests that some types of motion may have periodic components that can be fit to a model, such as a frequency domain model for example. In some embodiments, the position and orientation data are fit to a model and the position and orientation data reconstructed in accordance with the model.

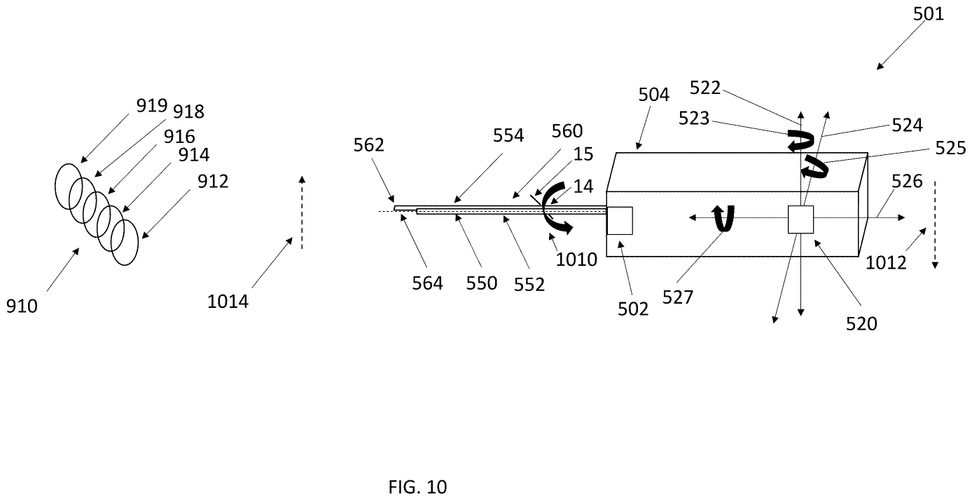

FIG. 10 shows a probe apparatus 501 comprising a sensor pivoting 520 about an opening such as an incision 14. In some embodiments, the probe is inserted through an incision in tissue such as corneal tissue 15, although the probe can be inserted through any suitable opening such as an opening in tissue or an opening in any material sized to receive the probe 560. In some embodiments, movement of the proximal portion of the probe apparatus 501 such as movement of a handpiece comprising the housing 504 results in a pivot 1010 about the opening 14. In some embodiments, movement of the housing 504 in a first direction 1012 on a first side of the pivot 1010 results in movement in a second direction 1014 on the second side of the pivot, which is opposite the first direction. For example translation of the housing 504 along first axis 522 or second axis 524 may result in an opposite movement of the probe tip on the second side of the pivot. In some embodiments, the pivot 1010 comprises a two dimensional pivot about the incision 14 associated with translation along axis 522 and axis 524, for example with reference to translation and rotation of the handpiece comprising housing 504. In some embodiments, translation of the probe 560 along axis 526 results in translation of the probe tip along incision 14 without pivoting. In some embodiments, rotation 527 of the probe 560 around axis 526 results in corresponding rotation of the imaging channel 552 and treatment channel 554.

The probe apparatus 501 may comprise any suitable probe apparatus as described herein, for example with reference to FIGS. 5A to 6C. In some embodiments, the processor is configured to measure a plurality of measurement or imaging beam locations 910 in response to movement about the pivot, such as a first measurement or imaging beam location, 912, a second measurement or imaging beam location 914, a third measurement or imaging beam location 916, a fourth measurement or imaging beam location 918 and a fifth measurement or imaging beam location 919, in which the plurality of measurement or imaging beam locations move in a second direction 1014 opposite a first movement direction 1012 of the handpiece comprising housing 504.

In some embodiments, the processor is operatively coupled to the sensor 520 and configured to detect the movement of the probe tip in response to the pivot. In some embodiments, the elongate probe 560 is coupled to the imager 502 and the elongate probe is sized for insertion into the opening 14. The sensor 520 is configured to measure an orientation of the probe and a translation of the probe, and the processor is configured to detect a pivoting of the elongate probe about the opening 14. In some embodiments, the processor is configured to determine a position and orientation of a measurement or imaging beam from the imaging device 502 in response to the elongate probe pivoting about the opening 14. In some embodiments, the processor is configured to determine a position and an orientation of a tip 562 of the probe 560 from sensor data in response to the probe pivoting about the opening 14.

In some embodiments, the housing 504 comprises a handpiece coupled to the elongate probe 560, the imaging device 502 and the sensor 520, and the processor is configured to determine one or more of a position or an orientation of a tip of the probe in response to a movement of the handpiece opposite a movement of the tip of the probe.

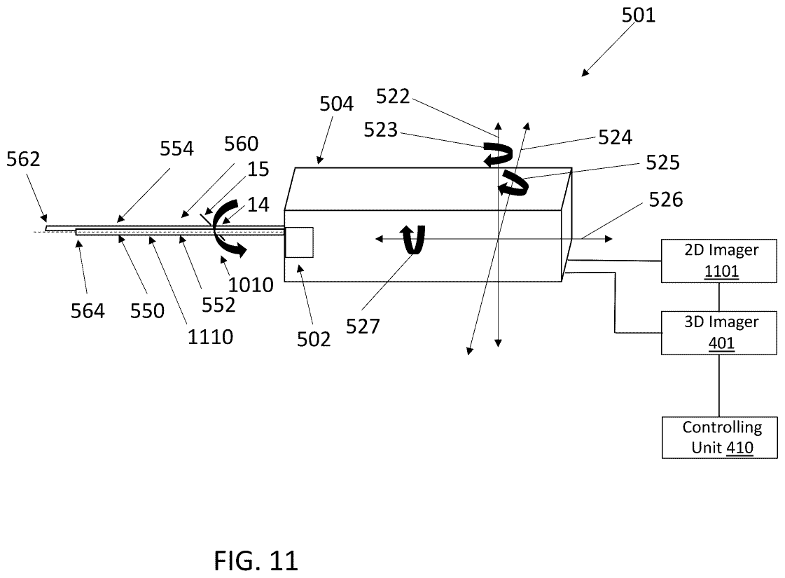

FIG. 11 shows a probe apparatus 501 in which the probe 560 comprises an endoscope 1110 configured to determine locations of a measurement or imaging beam of a 3D imaging device 502. In some embodiments, the imaging channel 552 comprises the endoscope and the 3D imaging device 502. The imaging channel 552 is coupled to a 2D imager 1101 such as a sensor array, and to one or more components of the 3D imager 401 as described herein. In some embodiments, images from endoscope 1110 are used to determine the position of the measurement or imaging beam of the 3D imager. In some embodiments, the probe 560 comprises a treatment channel 554 as described herein. While the probe apparatus 501 can be configured in many ways, in some embodiments the probe 560 is sized to fit within opening 14 through tissue such as corneal tissue 15. In some embodiments, the probe apparatus 501 is configured to pivot about opening 14. Alternatively or in combination, the probe apparatus 501 can be configured to perform measurements without being inserted through opening 14, for example in a free hand configuration.

The imaging channel 552 of probe 560 can be configured with endoscope 1110 in many ways and may comprise one or more of an adjacent configuration, a parallel configuration, an overlapping configuration, a coaxial configuration or a concentric configuration, for example. The endoscope 1110 of probe apparatus 501 can be combined with any 3D imaging device as described herein, for example with reference to FIGS. 5A to 6C. The endoscope 1110 can be configured in many ways and may comprise one or more lenses to image tissue, and may comprise an array of optical fibers such as an ordered array of optical fibers. In some embodiments, the endoscope comprises a sensor array on one or more of the probe 560, within the housing 504, or within a console of a treatment station, for example.

The sensor array of the two dimensional (2D) imager may comprise any suitable sensor array capable of generating a 2D image, such as one or more of a charge coupled device (CCD) array, a complimentary metal oxide semiconductor (CMOS) array, or other 2D sensor array as will be understood by one of ordinary skill in the art.

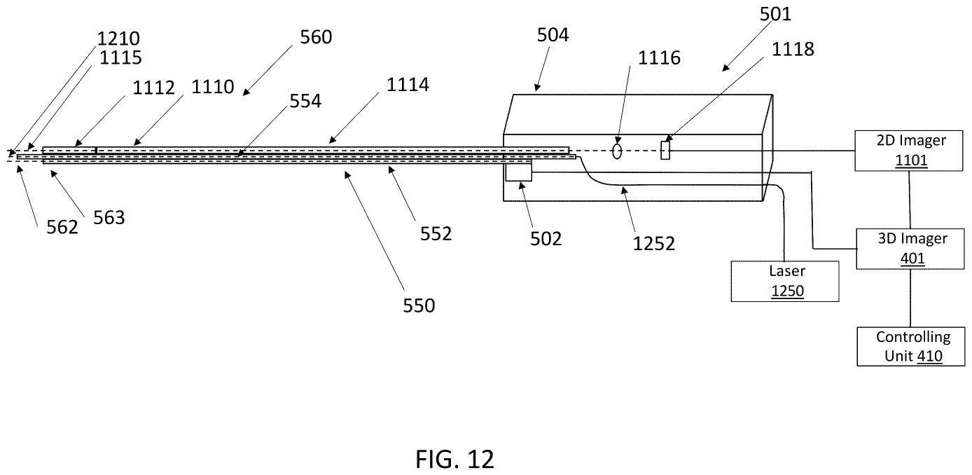

FIG. 12 shows a probe apparatus 501 in which probe 560 comprises a treatment channel 554, and an imaging channel comprising a 3D imager component and an endoscope 1110 to determine a location of the measurement or imaging beam of the 3D imager. In some embodiments, the endoscope 1110 comprises a sensor array 1118 upon which an image of tissue is formed. The sensor array can be located on probe 560, within housing 504 of the handpiece, or on an external console coupled to controlling unit 410, for example. In some embodiments, the sensor array 1118 is coupled to the endoscope 1110 with one or more optical fibers, such as an ordered array of optical fibers, for example.

While the treatment channel 554 can be configured in many ways as described herein, in some embodiments the treatment channel 554 comprises an optical fiber 1252 that extends to a distal tip 562 of probe 560. In some embodiments, the distal tip 562 of the treatment channel 554 extends beyond a distal tip 563 of the imaging channel 552, in order to simultaneously image the distal tip 562 of the treatment channel and tissue as described herein. In some embodiments, the treatment channel 554 comprises a treatment axis 1210, such as a propagation medium for optical energy or one or more of a tube or elongate element to deliver an implant, for example. The optical fiber 1250 is coupled to a laser 1250. The laser may comprise any laser configured to emit treatment light energy, such as one or more of ultraviolet light, visible light, near infrared light or visible light, for example. In some embodiments, laser 1250 comprises a Xenon Chloride excimer laser, for example. In some embodiments, the imaging channel 552 comprises one or more optical fibers or components of an ultrasound imaging probe as described herein. In some embodiments, the imaging channel 552 comprises a first channel for the 3D measurement or imaging beam and a second channel for an endoscope to perform 2D measurements to determine the position of the measurement or imaging beam. The treatment channel 554 and the one or more imaging channels can be arranged in any suitable way as described herein, such as a side by side configuration or an adjacent configuration, for example. In some embodiments, the imaging channel 552 of imaging device 502 extends along an imaging axis 564 and the endoscope 1110 extends along an endoscope axis 1210. The field of view of the imaging device 502 and the field of view of the endoscope 1110 can be configured to overlap, to allow the position of the measurement or imaging beam of the 3D imaging device to be determined as described herein.

While the endoscope 1110 can be configured in many ways, in some embodiments, endoscope 1110 comprises a first lens 1112 such as a gradient index (GRIN) lens and a second lens 1116 with a propagation portion 1114 in between. The second lens 1116 may comprise any suitable lens such as a camera lens or a GRIN lens, for example. The propagation portion 1114 may comprise any suitable optical transmission medium such as air or one or more optical fibers, for example.

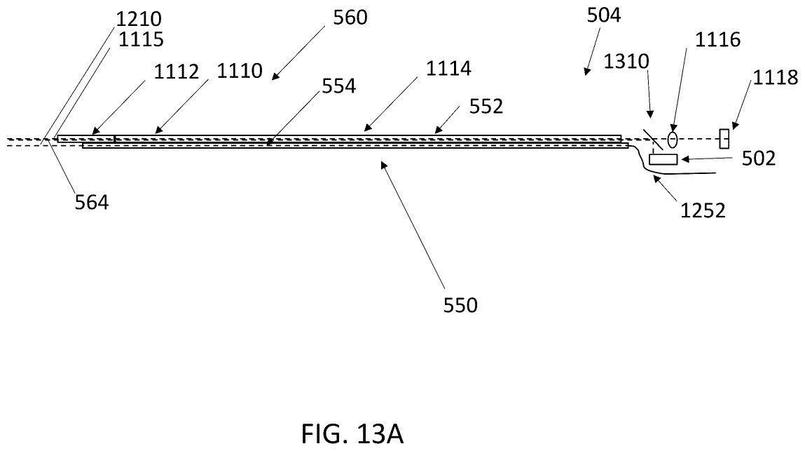

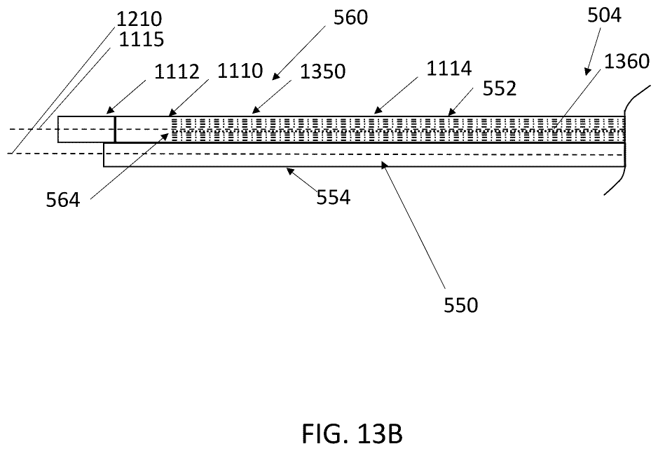

FIG. 13A shows a probe comprising a treatment channel 554, and 2D and 3D imaging components comprising an overlapping optical path along imaging channel 552. In some embodiments, one or more components of imaging device 502 is optically coupled to one or more components of endoscope 1110, such that the endoscope optical path along axis 1115 and the optical path of the 3D imaging device 502 overlap. While the endoscope 1110 can be optically coupled to the 3D imaging device 502 in many ways, in some embodiments a beam splitter 1310 is used to couple the endoscope to the 3D imaging device. Alternatively or in combination, optical fibers with couplers can be used to couple the imaging device 502 to the endoscope 1110. The beam splitter 1310 can be located at any suitable location along the optical path of the endoscope, such as distal to lens 1116 as shown or proximal to lens 1116, for example. In some embodiments, the 3D measurement or imaging beam along imaging axis 564 is substantially coaxial with optical path of the endoscope along axis 1115 in order to decrease parallax of the measurement or imaging beam location determined from endoscope images as described herein.

FIG. 13B shows a probe 560 comprising a treatment channel 554, a 3D imaging optical fiber 1360 and a plurality of 2D imaging optical fibers 1350 extending along imaging channel 552. In some embodiments, the plurality of 2D imaging optical fibers 1350 is located around the 3D imaging optical fiber 1360, which can decrease parallax of the location of the 3D measurement or imaging beam determined from the endoscope images as described herein. In some embodiments, the imaging optical fiber 1360 is located near a center of the plurality of imaging optical fibers near axis 1210 of endoscope 1115 of endoscope. The plurality of endoscopic imaging optical fibers 1350 can be arranged with respect to the 3D imaging optical fiber 1360 in many ways, for example with an annular array or a hexagonal array extending around 3D imaging optical fiber 1350.

In some embodiments, the lens 1112 is configured to form an image of the tissue on the distal ends of the plurality of imaging optical fibers 1350. In some embodiments, the lens 1112 is configured for project the measurement or imaging beam from the 3D imaging optical fiber 1360 onto the tissue and to image light from the tissue at the location of the measurement or imaging beam into the 3D imaging optical fiber 1360. The light collected with the 3D imaging optical fiber is transmitted to the 3D imaging device as described herein.

In some embodiments, the light from the plurality of imaging optical fibers is provided to a sensor array of the endoscope, for example by imaging the proximal ends of the plurality of optical fibers onto a sensor array with a second lens so as to provide an endoscopic image on the sensor array. The sensor array can be located on the probe 560, within the housing of the handpiece, or within a console of a treatment system. In some embodiments, the plurality of imaging optical fibers 1350 extends from the handpiece to a console of a treatment system such as a laser treatment system and is coupled to the sensor array with a connector. The plurality of optical fibers 1350 may comprise an ordered array of optical fibers so as to transmit the image formed on the proximal ends of the fibers to the sensor array, for example.

The imaging channel and treatment channel can be configured in many ways. In some embodiments, there is parallax between the axis of the imaging channel and the treatment channel. In some embodiments, there is parallax between the imaging channel and the treatment channel, and a processor is configured to adjust the constructed 3D image in response to a parallax angle and a distance between the distal end of the imaging channel and the distal end of the treatment channel, such as an implant or treatment fiber. In some embodiments, the processor is configured to adjust a position of the 3D constructed image in response to a distance between the distal end of the 3D imaging channel and the tissue. In some embodiments, the processor is configured to adjust the 3D image of one or more of the tissue or the distal end of the treatment channel with an artificial intelligence (AI) algorithm, for example.

The treatment channel and the imaging channel can be arranged on the probe in many ways. In some embodiments, the 3D imaging channel is located on an upper side of the probe, and the treatment channel is located on a lower side of the probe, for example with reference to the imaging probe 25 and the treatment probe 23 shown in FIG. 3. Alternatively, the 3D imaging channel can be located on a lower side of the probe, for example as shown with reference to FIGS. 5A to 6C and 9 to 13B. Although each of these figures show the imaging probe on a lower side of the probe, the imaging can probe can be located above the treatment probe as shown in FIG. 3, as will be understood by one of ordinary skill in the art. Alternatively, the imaging probe and the treatment probe may be arranged laterally with respect to each other, such that the elongate axes of the probes are located substantially laterally to each other, e.g. to within 10 degrees of horizontal from each other, for example.

Each of the probes as described herein may comprise a single use sterile disposable probe. In some embodiments, the sterile probe and housing comprising the handpiece are contained within a sterile sealed packaging. In some embodiments, the sensor array comprises a single use sensor array within the housing comprising the handpiece as described herein. In some embodiments, the sensor array comprises a sterilized sensor array, or a sterilizable sensor array, and combinations thereof, for example.

Although reference is made to probes such as hand held probes, the probes described herein can be used with surgical robotics systems such as surgical robotics systems comprising robotic arms. The treatment channel as described herein can be configured in many ways, such for the placement of implants and creating openings in the trabecular meshwork, for example with one or more of tissue manipulation, incision, or ablation. In some embodiments, the treatment channel comprises an end effector, such as an end effector of a surgical robotic system configured to manipulate tissue. The end effector may comprise any suitable end effector such as a blade or forceps, for example.

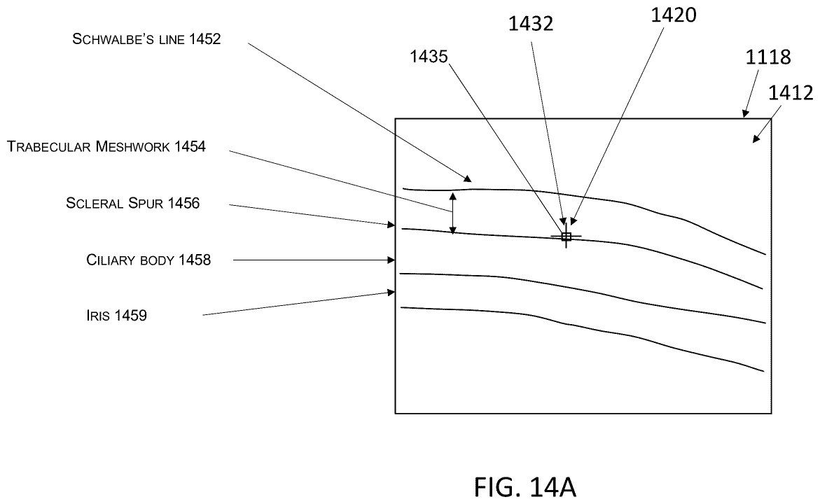

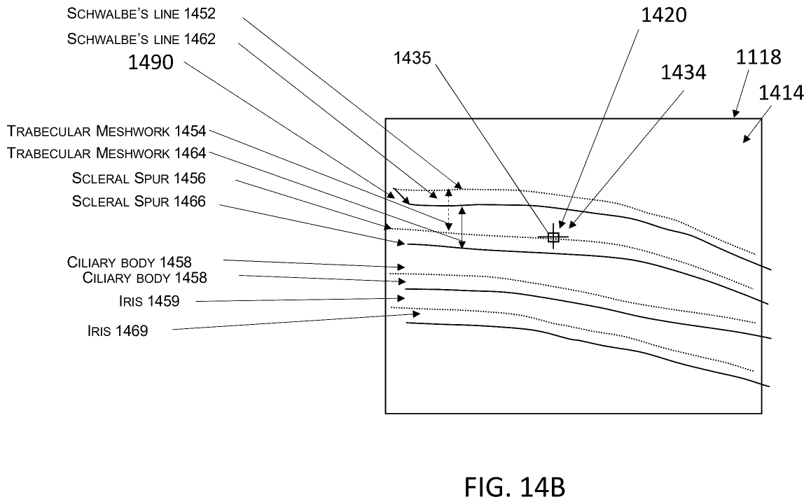

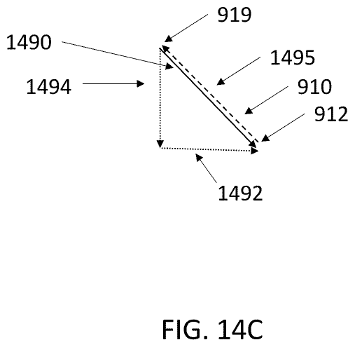

FIG. 14A shows an image 1412 such as an endoscopic image comprising one or more tissue structures and a location 1432 of a measurement or imaging beam 1420 at a first time on a sensor array 1118. In some embodiments, the location of the measurement or imaging beam 1420 in the image corresponds to one or more pixels 1435, such as a group of pixels in the image. In some embodiments, the one or more pixels 1435 are used as a reference location of the measurement or imaging beam. In some embodiments, the sensor array 118 is aligned with the measurement or imaging beam, such that the location of the one or more pixels 1435 corresponds to the location 1432 of the measurement or imaging beam. The change in the position of the tissue results in a change in position of the measurement or imaging beam on the tissue, which can be used to construct the 3D image. The one or more tissue structures of the image may comprise any suitable tissue structure such as one or more of a Schwalbe's line, a trabecular meshwork, a scleral spur, a ciliary body, an iris, or a Schlemm's canal, for example. In some embodiments a first image comprises a first location of the one or more tissue structures and a second image comprises a second location of the one or more tissue structures. In some embodiments, a first image comprises one or more of a first location 1452 of Schwalbe's line, a first location 1454 of the trabecular meshwork, a first location 1456 of the scleral spur, a first location 1458 of the ciliary body, or a first location 1459 of the iris.

FIG. 14B shows a second endoscope image 1414 comprising one or more tissue structures and a second location 1434 of a measurement or imaging beam 1420 at a second time. Although the second location 1434 of the measurement or imaging beam 1420 and the one or more pixels 1435 remain at a fixed location of sensor array 1118, the location of the one or more tissue structures can change between the first image 1412 and the second image 1414. The one or more tissue structures can be offset between the first image and the second image with one or more of a rotation or a translation between the first image and the second image. In some embodiments, the tissue structures shift between the first image and the second image with a movement vector 1490. In some embodiments, a second image comprises one or more of a second location 1462 of Schwalbe's line, a second location 1464 of the trabecular meshwork, a second location 1466 of the scleral spur, a second location 1468 of the ciliary body, or a second location 1469 of the iris. The locations of the corresponding one or more tissue structures from the first image are shown with dashed lines.

FIG. 14C shows an image displacement vector 1490 and a plurality of corresponding locations 910 of the measurement or imaging beam. In some embodiments, the displacement vector 1490 comprises a first component 1492, such as an X displacement along the tissue and a second displacement 1494 such as a Y displacement along the tissue. The displacement of the measurement locations relative to the tissue is shown with vector 1495, and is generally opposite the movement of the tissue shown in the images. In some embodiments, a plurality of measurements of the 3D imaging beam are obtained between the first image and the second image, and the values can be interpolated. For example, if the first image corresponds to a first measurement or imaging beam location 912 and the second image corresponds to a fifth measurement or imaging beam location 919, the second, third and fourth measurement or imaging beams can be interpolated to intermediate locations along the displacement vector.

In some embodiments, the processor is configured to receive a plurality of 2D images of the tissue and determine a position of the measurement or imaging beam for each of the plurality of 2D images and construct the 3D image in response to said each of the plurality of 2D images. In some embodiments, processor is configured to assign a location of the measurement or imaging beam for each of a plurality of A-scans in response to the plurality of 2D images. The A-scan sampling rate may be faster than the frame rate of the sensor array. In some embodiments, the processor is configured to interpolate the location of the measurement or imaging beam for each of the plurality of A-scans in response to the plurality of 2D images. For example, the A-scan imager can be configured to sample the plurality of A-scans at a sample rate that is at least 100 times faster than a frame rate of the sensor array. In some embodiments, each of the plurality of 2D images comprises a tissue structure and response to the position of the measurement or imaging beam away from the tissue structure.

In some embodiments, the processor is configured to detect a tremor of a user in and construct a tremor model in response to the movement of the tissue structure among the plurality of 2D images.

While FIGS. 14A to 14C make reference to probe movement, in some embodiments the movement of the images corresponds to tissue movement as described herein, such as periodic movement, for example. In some embodiments, movement of the tissue as shown in FIGS. 14A to 14C is related to pulsatile flow of blood, which can result in movement of one or more of the Schlemm's canal, the collector channels, the trabecular meshwork or the retina. Although reference is made to images of anterior tissues of the eye, any tissue described herein can be imaged, such as retinal tissue, for example.

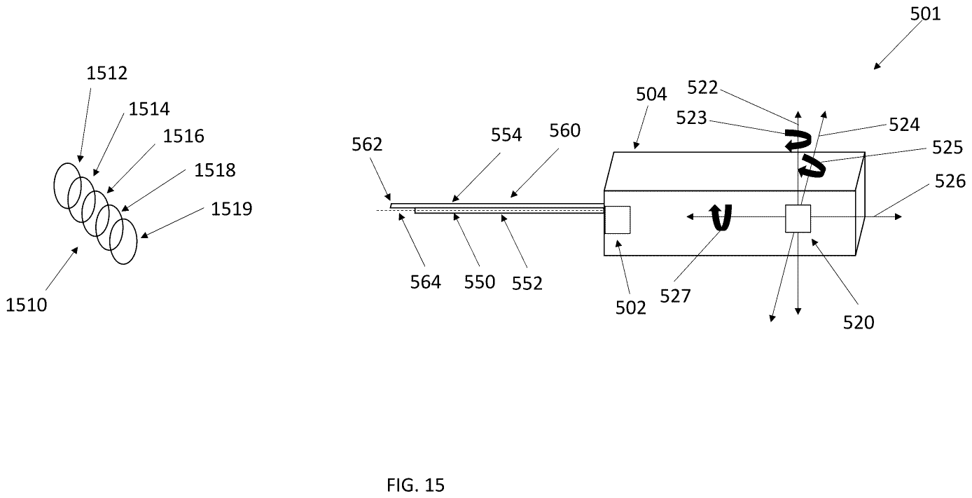

FIG. 15 shows probe 560 and corresponding measurement locations 1510 of a measurement or imaging beam in response to tissue movement such as periodic tissue movement as described herein. In some embodiments, movement of the tissue results in the measurement or imaging beam sampling tissue at a plurality of locations 1510 while the measurement or imaging beam remains substantially fixed, such as may result from movement of the tissue, which may comprise periodic movement of the tissue as described herein. The position and orientation of the probe 560 and probe apparatus 501 may remain substantially fixed for example. Alternatively or in combination, data related to the movement of the tissue may be combined with probe movement data as described herein. The plurality of locations of the measured tissue may comprise a first location 1512 corresponding to a first position of the tissue,, a second position 1514 corresponding to a second location of the tissue, a third position 1516 corresponding to a third location of the tissue, a fourth location 1518 corresponding to a fourth location of the tissue, and a fifth location 1518 corresponding to a fifth location of the tissue. In some embodiments, each of the plurality of locations corresponds to a measurement location of the beam during a scan such as an A-scan. The position can be determined with data such as sensor data or movement from an endoscopic image, for example. In some embodiments, tissue movement data is combined with the position and orientation of the probe measured for each of the plurality of A-scans. The position and orientation data may comprise position and orientation data measured for each of the first axis 522, the second axis 524, and the third axis 526 as described herein for example.

In some embodiments, a plurality of position and orientation measurements are fit to a model. Work in relation to the present disclosure suggests that some types of motion may have periodic components that can be fit to a model, such as a frequency domain model for example. In some embodiments, the position and orientation data are fit to a model and the position and orientation data reconstructed in accordance with the model.

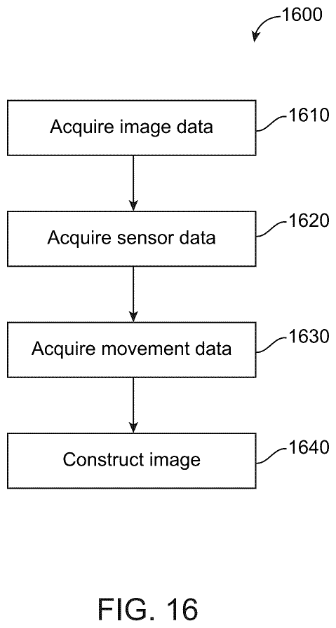

FIG. 16 shows a method 1600 of imaging tissue.

At a step 1610, image data is acquired with an imager coupled to a sensor configure to measure one or more of a position or an orientation of the imager.

At a step 1620, sensor data is acquired from the sensor.

At a step 1630 movement data is acquired. The movement data may be acquired in any suitable way and may comprise movement data generated or acquired from one or more of the sensor data or the imager data. The movement data may comprise periodic movement data such as periodic movement related to one or more of tremor, resonance or pulsatile tissue. In some embodiments, the periodic movement corresponds to harmonics, which can be determined in response to the movement data. In some embodiments, the periodic movement corresponds to pulsations of tissue, such as the choroid under the retina. The movement data can be used to construct the 3D image.

In some embodiments, the periodic movement is determined with a sensor configured to measure a cardiac cycle of the tissue of the patient, such as a cardiac cycle of the patient. In some embodiments, the processor is configured to construct the 3D image in response to the cardiac cycle. While the cardiac signal can be measured in many ways, in some embodiments, the sensor comprises one or more of an electrocardiogram (EKG) sensor, a pulse oximeter, or a blood oxygen sensor.

At a step 1640, an image such as a 3D image is constructed in response to the image data and the sensor data.

Although FIG. 16 shows a method 1600 of imaging tissue in accordance with some embodiments, a person of ordinary skill in the art will recognize many adaptations and variations. For example, the steps may be performed in any order. Some of the steps may be omitted, and some of the steps repeated. Some of the steps may combine sub steps of other steps.

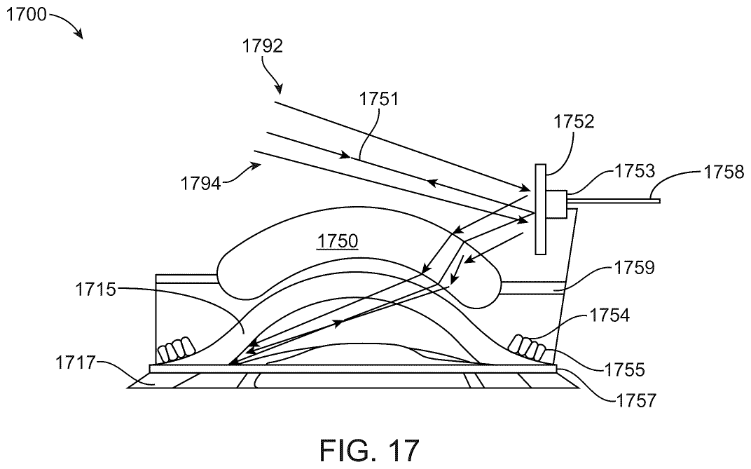

FIG. 17 shows a femtosecond laser and OCT system 1700 comprising a sensor. An indirect goniolens 1750 attached to the sclera 1717 mechanically or by prongs 1756 or suction, with an internal mirror 1752. The mirror may be individual or segmented and fixed or mobile to enable scanning for both viewing and for treatment targeting. In the condition of a mobile mirror/mirror surface, the mirror 1752 can be controlled mechanically or pneumatically or with a Mylar type surface reflecting balloon. The mirror can be plano, concave, convex and singular or in a segmented array.

In some embodiments, the system 1700 comprises a plurality of laser beams, such as a first laser beam 1792 and a second laser beam 1794. The laser beam may comprise visible laser beams, such as red laser beams, for example from one or more red laser diodes. The laser beams can be configured to substantially overlap at the tissue to be treated, such that the beams appear as a single beam at the target tissue such as the trabecular meshwork or Schlemm's canal, for example. If the tissue is not positioned at the correct distance from the optical path, the overlap of the beams decreases and the beams may appear as separate spots on the target tissue.

The system 1700 may comprise additional components, such as the processor and arrays as described herein, for example with reference to FIG. 7. For example, the system 1700 may comprise the operating microscope, display, the 3D imager, the controlling unit 400, and a femtosecond laser unit.

Examples of laser delivery systems and femto second lasers suitable for incorporation in accordance with the present disclosure are described in U.S. application Ser. No. 14/732,627, filed on Jun. 5, 2015, “entitled Methods and apparatuses for the treatment of glaucoma using visible and infrared ultrashort laser pulses”, published as US20160095751 on Apr. 7, 2016, the entire disclosure of which has been previously incorporated herein by reference.

Referring again with reference to FIG. 7, in conjunction with the optical microscope 409, an imaging device coupled to probe apparatus 501 as described herein may provide a data to 3D imager 401 and the controlling unit 410, such as the overlap of the beams 1792 and 1794 on the trabecular meshwork. In some embodiments, a second camera 416 comprising a detector array is optically coupled to the microscope 409 to receive optical images from the operating microscope 409, and optically coupled to the processor 414 of the control unit 410, and the overlap of the beams can be determined. The control unit 410 can receive the image data from the camera 416 and process the image data to provide visual image data on the heads-up display 407 and overlay the visual image data on an anterior optical image of the operating microscope 409, such as an anterior image showing the beams 1792 and 1794, which may overlap at the target tissue such as the trabecular meshwork.

Referring again to FIGS. 14A and 14B, the image of the tissue and the beams 1792 and 1794 illuminating the tissue may appear on the two dimensional sensor array, similar to the two dimensional endoscope array 1118 shown in FIG. 14. In some embodiments, the two dimensional sensor array comprises a two dimensional sensor array of a camera 416. The of the operating microscope, and the separation distance of the beams can be used to determine the position of the tissue along the optical path of the imaging beam, and in some embodiments the position along the beam 1751. The one or more pixels 1435 shown in FIGS. 14A and 14B can be used to determine the position of the measurement beam, and the separation distance between the first beam 1792 and the second beam 1794 can be used to determine the position of the tissue such as the trabecular meshwork along the optical axis, in order to determine the position of the tissue in three dimensions, for example along the optical axis of the measurement beam and transverse to the measurement beam, as described herein for example with reference to FIGS. 14A, 14B and 17.

In some embodiments, the processor is configured to image the tissue with a plurality of scanning A-scans and measure the separation distance for each of the plurality of A-scans, and the processor is configured to construct the image in response to each of the plurality of A-scans and the separation distance for each of the plurality of A-scans. In some embodiments, the processor is configured to construct the image in response to each of the plurality of A-scans, the plurality of separation distances, and the transverse position of the tissue as described herein, for example with reference to FIGS. 9, 10, 14A and 14B.

In some embodiments, a beam 1751 of pulsed radiation is generated by a femtosecond laser and delivered into the eye by the delivery system, including the goniolens 1750. In some embodiments, the beam 1751 comprises a bidirectional beam, in which the OCT beam is directed to the tissue and receives light from the tissue where the OCT beam is focused. In some embodiments, the OCT beam comprises sufficient resolution to measure a separation distance between an inner wall of Schlemm's canal and an outer wall of Schlemm's canal.

In some embodiments, the beam 1751 comprises a plurality of beams. In some embodiments, the beam 1751 comprises a femto second laser beam and an imaging beam as described herein. Alternatively, the imaging beam may comprise a separate beam directed to the tissue. The imaging beam may comprise any imaging beam as described herein. In some embodiments, the processor of the system is configured to scan a plurality of A-scans of the imaging beam along the tissue such as the trabecular meshwork to image the tissue.

In some embodiments, the beam 1751 comprises a plurality beams, such as an OCT imaging beam and a treatment laser beam, for example a bidirectional laser beam. Although shown as substantially coaxial, in some embodiments the treatment beam and the imaging beam are offset from each other while approaching the eye, similarly to the first laser beam 1792 and the second laser beam 1794.

The beam 1751 is reflected by a mirror 1752 which may be controlled by a servo system 1753 connected to a controller 1758 to focus scanning photodisruptive energy onto the curved surface of the target tissue. The optics enable bidirectional use, one direction is used to treat the target tissue, the other direction is used to view and/or sense the x, y, z coordinates of the targeted tissue to enable precise treatment and removal of the target regions. The beam 1751 has a set of pulse parameter ranges specifically selected to photodisrupt targeted tissue of the trabecular meshwork, while minimizing damage to surrounding tissue. Thus, the beam has a wavelength between 0.4 and 2.5 microns. The exact wavelength used for a particular subject depends on tradeoffs between strong absorption by the meshwork and transmission of preceding ocular structures and aqueous humor.

In some embodiments, an indirect goniolens 1750 is coupled to the cornea 1715 or by suction and coupled to an internal mirror 1752. In some embodiments a goniolens is coupled to the sclera 1717 by suction 1757 or mechanically with a mirror system 1752 external to the goniolens.

In some embodiments, the pulse duration of the laser beam is chosen to have a high probability of photodisrupting material of the corneoscleral angle outflow tissues. In some embodiments, there is an inverse relationship between the laser pulse duration and the energy required in each pulse to generate optical breakdown. In some embodiments, the pulse duration is selected to be shorter than the thermal relaxation of the target so that only the targeted material is heated and the surrounding tissue is unaffected. Thus, the pulse duration is between 20 fs and 300 ps. The pulse rate is between 1 and 500 KHz

In some embodiments, the pulse energy is chosen to facilitate photodisruption and minimize the shockwave effect of the laser light. A typical value for the pulse energy is between 300 to 1500 nJ.

In some embodiments, the spot diameter is chosen such that sufficient laser energy density is provided to facilitate photodisruption of one or more tissues, such as one or more the trabecular meshwork, the juxtacanalicular trabecular meshwork. In some embodiments, the spot size is between 1 to 10 microns.

In some embodiments, the goniolens 1750 is anchored either on the sclera 17 or the cornea 15 by a suction ring 57 or prongs 56, for example. The anchoring system is attached to a pressure regulating system 55 and an ocular pulse sensing system 54. The anchoring system is either concentric 57 or segmented 56. Scanning the spot in the x, y, and z direction effects patterns for tissue removal. Although reference is made to a gonio lens, the lens coupled to the eye may comprise any suitable lens shaped to receive the cornea, and the lens may comprise on or more components of a patient interface. The mirror can be coupled to the lens in many ways. In some embodiments, the lens comprises an external mirror located outside of the goniolens. Alternatively or in combination, the lens may comprise an internal mirror located within the goniolens. Examples of suitable lens and mirror combinations are described in FIGS. 4, 5 and 6 of U.S. application Ser. No. 14/732,627, filed on Jun. 5, 2015, “entitled Methods and apparatuses for the treatment of glaucoma using visible and infrared ultrashort laser pulses”, published as US20160095751 on Apr. 7, 2016, the entire disclosure of which has been previously incorporated herein by reference.

Although reference is made to detection of the cardiac cycle with an EKG, pulse oximeter or oxygen sensor, in some embodiments, the OCT system comprises sufficient resolution to detect pulsation of the tissue, for example widening and narrowing of Schlemm's canal. In some embodiments, the OCT system comprises sufficient resolution to determine changes in distance between an inner wall and an outer wall of Schlemm's canal with a plurality of A-scans as described herein. In some embodiments, the distance across Schlemm's canal increases and decreases in response to the cardiac cycle, and the OCT system is configured to measure the change in the distance with the plurality of A-scans, for example.

As detailed above, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. For example, one or more of the devices recited herein may receive image data of a sample to be transformed, transform the image data, output a result of the transformation to determine a process, use the result of the transformation to perform the process, and store the result of the transformation to produce an output image of the sample. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

A processor as described herein can be configured to perform one or more steps of any method described herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising”.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

The present disclosure includes the following clauses.
Clause 1. An apparatus to image tissue, comprising: an imager configured to generate an imaging beam to image the tissue; a sensor coupled to the imager to acquire sensor data related to one or more of a position or an orientation of the imaging beam; a processor coupled to the imager and the sensor to acquire image data and sensor data, the processor configured with instructions to construct a 3D image of the tissue in response to the image data and the sensor data.
Clause 2. The apparatus of the preceding clause, wherein the imaging beam comprises one or more of an optical imaging beam, an optical coherence tomography (OCT) imaging beam, an ultrasound (US) imaging beam or a photoacoustic imaging beam.
Clause 3. The apparatus of any of the preceding clauses, wherein the imager comprises the sensor, the sensor configured to generate movement data, the processor configured to reconstruct the 3D image in response to the movement data.
Clause 4. The apparatus of any of the preceding clauses, wherein the optical imager comprises a two dimensional imager comprising a two dimensional sensor array, the two dimensional imager comprising one or more of an endoscope, a microscope, a stereoscopic microscope, a stereoscopic endoscope.
Clause 5. The apparatus of any of the preceding clauses, wherein the imager comprises an imaging axis and emits the beam along the axis and the sensor comprises a plurality of axes to measure the one or more of the position or the orientation of the beam of imaging energy.
Clause 6. The apparatus of any of the preceding clauses, wherein the axis of the beam is aligned with respect to at least one of the plurality of axes to measure the one or more of the position or the orientation beam of imaging energy.
Clause 7. The apparatus of any of the preceding clauses, wherein the sensor is configured to measure a movement of the imager in a direction along the axis of the beam corresponding to a movement of the imaging beam along the axis and the processor is configured to construct the 3D image in response to the movement of the imaging beam along the axis.
Clause 8. The apparatus of any of the preceding clauses, wherein the sensor is configured to measure a movement of the imager transverse to the axis of the imaging beam corresponding to a movement of the imaging beam transverse to the axis and the processor is configured to construct the 3D image in response to the movement of the imaging beam transverse to the axis.
Clause 9. The apparatus of any of the preceding clauses, wherein the movement of the imaging beam transverse to the axis corresponds to a movement of the imaging beam in a first direction transverse to the axis and a second direction transverse to the axis and wherein the processor is configured to construct the 3D image in response to the movement of the imaging beam in the first direction and the second direction.
Clause 10. The apparatus of any of the preceding clauses, wherein the movement of the imager comprises a rotation of the imager corresponding to the movement of the imaging beam.
Clause 11. The apparatus of any of the preceding clauses, wherein the movement of the imager comprises a translation of the imager corresponding to the movement of the imaging beam.
Clause 12. The apparatus of any of the preceding clauses, wherein the first direction transverse to the axis and the second direction transverse to the axis defines a plane transverse to the axis and optionally wherein the plane extends substantially perpendicular to the axis.
Clause 13. The apparatus of any of the preceding clauses, wherein the processor is configured to store a plurality of positions and a plurality of orientations of the imager while the beam of imaging energy is directed to the tissue and to construct the 3D image in response to the plurality of positions and orientations.
Clause 14. The apparatus of any of the preceding clauses, wherein the processor is configured to determine a plurality of locations of the imaging beam from the sensor data and wherein the processor is configured to construct the 3D image from the plurality of positions and orientations of the sensor.
Clause 15. The apparatus of any of the preceding clauses, further comprising: an elongate probe coupled to the imager, the elongate probe sized for insertion into an opening; wherein the sensor is configured to measure an orientation of the probe and a translation of the probe.
Clause 16. The apparatus of any of one the preceding clauses, further comprising: a handpiece coupled to the elongate probe, the imager and the sensor; wherein the processor is configured to determine one or more of a position or an orientation of a tip of the probe in response to a movement of the handpiece in a same direction as a movement of the tip of the probe.
Clause 17. The apparatus of any of the preceding clauses, further comprising a second probe, the second probe comprising elongate treatment probe, and optionally wherein the elongate treatment probe is not coupled to the elongate probe coupled to the imager and the sensor.
Clause 18. The apparatus of any of the preceding clauses, further comprising: an elongate probe coupled to the imager, the elongate probe sized for insertion into an opening; wherein the sensor is configured to measure an orientation of the probe and a translation of the probe; wherein the processor is configured to detect a pivoting of the elongate probe about the opening.
Clause 19. The apparatus of any of the preceding clauses, wherein the processor is configured to determine a position and orientation of the imaging beam from the imager in response to the elongate probe pivoting about the opening.
Clause 20. The apparatus of any of the preceding clauses, wherein the processor is configured to determine a position and an orientation of a tip of the probe from sensor data in response to the probe pivoting about the opening.
Clause 21. The apparatus of any of the preceding clauses, further comprising: a handpiece coupled to the elongate probe, the imager and the sensor; wherein the processor is configured to determine one or more of a position or an orientation of a tip of the probe in response to a movement of the handpiece opposite a movement of the tip of the probe.
Clause 22. The apparatus of any of the preceding clauses, wherein the sensor comprises a sensor array of an endoscope or a microscope and optionally wherein the sensor array comprises a two dimensional sensor array.
Clause 23. The apparatus of any of the preceding clauses, wherein the endoscope is configured to generate a two dimensional (2D) image the tissue with the sensor array and wherein the processor is configured to determine the position of the imaging beam in response to the 2D image of the tissue.
Clause 24. The apparatus of any of the preceding clauses, wherein the processor is configured to receive a plurality of 2D images of the tissue and determine a position of the imaging beam for each of the plurality of 2D images and construct the 3D image in response to said each of the plurality of 2D images.
Clause 25. The apparatus of any of the preceding clauses, wherein the processor is configured to construct the 3D image in response movement of the tissue structure among the plurality of 2D images.
Clause 26. The apparatus of any of the preceding clauses, wherein a position of the imaging beam in said each of the plurality of images is located away from the tissue structure and the processor is configured to construct the 3D image and wherein the processor is configured to assign a location of the imaging beam for each of a plurality of A-scans in response to the plurality of 2D images.
Clause 27. The apparatus of any of the preceding clauses, wherein the processor is configured to interpolate the location of the imaging beam for each of the plurality of A-scans in response to the plurality of 2D images.
Clause 28. The apparatus of any of the preceding clauses, wherein the imager is configured to sample the plurality of A-scans at a sample rate that is at least 100 times faster than a frame rate of the sensor array.
Clause 29. The apparatus of any of the preceding clauses, wherein said each of the plurality of 2D images comprises a tissue structure and response to the position of the imaging beam away from the tissue structure.
Clause 30. The apparatus of any of the preceding clauses, wherein the processor is configured to detect a movement of one or more of a user, a probe, a robotic arm or a tissue, and construct a movement model in response to the movement of the tissue structure among the plurality of 2D images.
Clause 31. The apparatus of any of the preceding clauses, wherein the movement model corresponds to periodic movement of the one or more of the user, the probe, the robotic arm or the tissue.
Clause 32. The apparatus of any of the preceding clauses, wherein the processor is configured to detect a tremor of a user and construct a tremor model in response to the movement of the tissue structure among the plurality of 2D images.
Clause 33. The apparatus of any of the preceding clauses, wherein the processor is configured to detect a movement of a tissue and construct a tissue movement model in response to the movement of the tissue structure among the plurality of 2D images.
Clause 34. The apparatus of any of the preceding clauses, wherein the movement model corresponds to movement of ocular tissue in response to pulsatile blood flow of a patient.
Clause 35. The apparatus of any of the preceding clauses, wherein the processor is configured to detect a resonance mode of a robotic arm and construct a resonance model in response to the movement of the tissue structure among the plurality of 2D images.
Clause 36. The apparatus of any of the preceding clauses, wherein the tissue structure comprises one or more of a Schwalbe's line, a ciliary body band, a scleral spur, a Schlemm's canal, a trabecular meshwork, or an iris of an eye.
Clause 37. The apparatus of any of the preceding clauses, wherein the imaging beam is aligned with one or more pixels of the sensor array and the processor is configured to construct the 3D image in response to locations of the one or more pixels in a plurality of 2D images.
Clause 38. The apparatus of any of the preceding clauses, wherein the one or more pixels is coaxially aligned with the imaging beam.
Clause 39. The apparatus of any of the preceding clauses, wherein the one or more pixels correspond to a reference location of the imaging beam.
Clause 40. The apparatus of any of the preceding clauses, wherein the imager is configured to generate a plurality of A-scans with the imaging beam, and the processor is configured to determine a position of the imaging beam for each of the plurality of A-scans in response to the sensor data and to construct the image in response to the plurality of positions of the imaging beam.
Clause 41. The apparatus of any of the preceding clauses, further comprising an imaging optical fiber, wherein the imager comprises one or more of the OCT imaging or the photoacoustic imaging and wherein an imaging beam is directed along the optical fiber.
Clause 42. The apparatus of any of the preceding clauses, further comprising an acoustic sensor located near a distal end of the optical fiber and wherein the imager comprises the photoacoustic imager, the sensor configured to receive an acoustic pulse in response to a imaging beam from the optical fiber illuminating the tissue to generate the acoustic pulse.
Clause 43. The apparatus of any of the preceding clauses, further comprising a scanner to scan the imaging beam.
Clause 44. The apparatus of any of the preceding clauses, wherein the scanner is configured to deflect a distal end portion of the imaging optical fiber to scan the imaging beam.
Clause 45. The apparatus of any of the preceding clauses, further comprising an ultrasound transducer to generate the plurality A-scans.
Clause 46. The apparatus of any of the preceding clauses, wherein the optical imager comprises one or more of an endoscope, a microscope, a stereoscopic microscope, a stereoscopic endoscope and the processor is configured to generate movement data from a plurality of images from the optical imager and the processor is configured to generate the 3D image of the tissue in response to the movement data and the plurality of images.
Clause 47. The apparatus of any of the preceding clauses, wherein the imager comprises the ultrasound (US) imager and the US imager comprises one or more arrays.
Clause 48. The apparatus of any of the preceding clauses, further comprising a treatment probe to treat the tissue, the treatment probe coupled to the sensor and the imager.
Clause 49. The apparatus of any of the preceding clauses, wherein the treatment probe comprises a treatment channel to treat the tissue.
Clause 50. The apparatus of any of the preceding clauses, wherein the treatment channel is configured to one or more of deliver an implant, deliver a treatment energy, or manipulate tissue.
Clause 51. The apparatus of any of the preceding clauses, wherein the treatment channel comprises an optical fiber to deliver laser energy to the tissue.
Clause 52. The apparatus of any of the preceding clauses, wherein the treatment channel comprises a working channel to deliver the implant to the tissue.
Clause 53. The apparatus of any of the preceding clauses, further comprising handpiece coupled to the imager and the sensor and wherein the processor is configured to detect a tremor of a user and construct a tremor model in response to the sensor data.
Clause 54. The apparatus of any of the preceding clauses, wherein the processor is configured to determine a position of the imaging beam in the tissue in response to the sensor data and the tremor model.
Clause 55. The apparatus of any of the preceding clauses, wherein the tremor corresponds to periodic movement of the handpiece.
Clause 56. The apparatus of any of the preceding clauses, wherein the imager is configured to emit an imaging beam and the processor is configured to determine a location of the imaging beam in response to the tremor detector with the sensor.
Clause 57. The apparatus of any of the preceding clauses, further comprising a sensor to measure a cardiac cycle of the tissue, wherein the processor is configured to construct the 3D image in response to the cardiac cycle.
Clause 58. The apparatus of any of the preceding clauses, wherein the sensor comprises one or more of an electrocardiogram (EKG) sensor, a pulse oximeter, or a blood oxygen sensor.
Clause 59. The apparatus of any of the preceding clauses, further comprising a plurality of visible laser beams to measure a position of the tissue along an optical path of the imaging beam.
Clause 60. The apparatus of any of the preceding clauses, wherein the sensor comprises a two dimensional sensor array and the processor is configured to determine the position of the tissue along the optical path in response to a separation distance between the plurality of visible laser beams on the sensor array.
Clause 61. The apparatus of any of the preceding clauses, wherein the processor is configured to construct the 3D image in response to the separation distance between the first laser beam and the second laser beam.
Clause 62. The apparatus of any of the preceding clauses, wherein the processor is configured to image the tissue with a plurality of OCT A-scans and to measure the separation distance for each of the plurality of A-scans.
Clause 63. The apparatus of any of the preceding clauses, wherein the processor is configured to construct the 3D image in response to the separation distance between the first measurement beam and the second measurement beam for each of the plurality of A-scans.
Clause 64. The apparatus of any of the preceding clauses, wherein the processor is configured to determine a transverse position of the measurement beam on the tissue in response to an image of the tissue on the two dimensional sensor array.
Clause 65. The apparatus of any of the preceding clauses, wherein one or more pixels of the two dimensional sensor array correspond to a location of the measurement beam in the image.
Clause 66. The apparatus of any of the preceding clauses, wherein the processor is configured to determine a three dimensional position of the measurement beam on the tissue in response to the separation distance between the first beam and the second beam and the separation distance between the first measurement beam and the second measurement beam.
Clause 67. The apparatus of any of the preceding clauses, further comprising a treatment laser, the treatment laser comprising one or more of an ultraviolet laser, a femto second laser, a visible laser or an infrared laser.
Clause 68. A method of imaging tissue, comprising: acquiring image data with an imager coupled to a sensor configure to measure one or more of a position or an orientation of the imager; acquiring sensor data from the sensor with the image data; constructing a 3D image of the tissue in response to the image data and the sensor data.
Clause 69. An apparatus to image tissue, comprising: an imager to image the tissue with one or more of an optical imager, an optical coherence tomography (OCT) imager, an ultrasound (US) imager or a photoacoustic imager; a processor coupled to the imager, the processor configured with a movement model, the processor configured with instructions to construct a 3D image of the tissue in response to the imager data and the movement model.
Clause 70. An apparatus or method of any of the preceding clauses, wherein a 3D imaging channel is located proximally to a distal end of a treatment channel to view one or more of an implant, an optical fiber, a tissue manipulator, or an end effector located on a distal end of the treatment channel.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. An apparatus to image tissue, comprising:
an imager configured to generate an imaging beam to image the tissue;
a sensor coupled to the imager to acquire sensor data related to one or more of a position or an orientation of the imaging beam;
a processor coupled to the imager and the sensor to acquire image data and sensor data, the processor configured with instructions to construct a 3D image of the tissue in response to the image data and the sensor data.

2. The apparatus of claim 1, wherein the imaging beam comprises one or more of an optical imaging beam, an optical coherence tomography (OCT) imaging beam, an ultrasound (US) imaging beam or a photoacoustic imaging beam.

3. The apparatus of claim 1, wherein the imager comprises the sensor, the sensor configured to generate movement data, the processor configured to reconstruct the 3D image in response to the movement data.

4. The apparatus of claim 1, wherein the optical imager comprises a two dimensional imager comprising a two dimensional sensor array, the two dimensional imager comprising one or more of an endoscope, a microscope, a stereoscopic microscope, a stereoscopic endoscope.

5. The apparatus of claim 1, wherein the imager comprises an imaging axis and emits the beam along the axis and the sensor comprises a plurality of axes to measure the one or more of the position or the orientation of the beam of imaging energy.

6. The apparatus of claim 5, wherein the axis of the beam is aligned with respect to at least one of the plurality of axes to measure the one or more of the position or the orientation beam of imaging energy.

7. The apparatus of claim 5, wherein the sensor is configured to measure a movement of the imager in a direction along the axis of the beam corresponding to a movement of the imaging beam along the axis and the processor is configured to construct the 3D image in response to the movement of the imaging beam along the axis.

8. The apparatus of claim 5, wherein the sensor is configured to measure a movement of the imager transverse to the axis of the imaging beam corresponding to a movement of the imaging beam transverse to the axis and the processor is configured to construct the 3D image in response to the movement of the imaging beam transverse to the axis.

9. The apparatus of claim 8, wherein the movement of the imaging beam transverse to the axis corresponds to a movement of the imaging beam in a first direction transverse to the axis and a second direction transverse to the axis and wherein the processor is configured to construct the 3D image in response to the movement of the imaging beam in the first direction and the second direction.

10. The apparatus of claim 9, wherein the movement of the imager comprises a rotation of the imager corresponding to the movement of the imaging beam.

11. The apparatus of claim 9, wherein the movement of the imager comprises a translation of the imager corresponding to the movement of the imaging beam.

12. The apparatus of claim 9, wherein the first direction transverse to the axis and the second direction transverse to the axis defines a plane transverse to the axis and optionally wherein the plane extends substantially perpendicular to the axis.

13. The apparatus of claim 5, wherein the processor is configured to store a plurality of positions and a plurality of orientations of the imager while the beam of imaging energy is directed to the tissue and to construct the 3D image in response to the plurality of positions and orientations.

14. The apparatus of claim 13, wherein the processor is configured to determine a plurality of locations of the imaging beam from the sensor data and wherein the processor is configured to construct the 3D image from the plurality of positions and orientations of the sensor.

15. The apparatus of claim 1, further comprising:
an elongate probe coupled to the imager, the elongate probe sized for insertion into an opening;
wherein the sensor is configured to measure an orientation of the probe and a translation of the probe.

16. The apparatus of claim 15, further comprising:
a handpiece coupled to the elongate probe, the imager and the sensor;
wherein the processor is configured to determine one or more of a position or an orientation of a tip of the probe in response to a movement of the handpiece in a same direction as a movement of the tip of the probe.

17. The apparatus of claim 15, further comprising a second probe, the second probe comprising elongate treatment probe, and optionally wherein the elongate treatment probe is not coupled to the elongate probe coupled to the imager and the sensor.

18. The apparatus of claim 1, further comprising:
an elongate probe coupled to the imager, the elongate probe sized for insertion into an opening;
wherein the sensor is configured to measure an orientation of the probe and a translation of the probe;
wherein the processor is configured to detect a pivoting of the elongate probe about the opening.

19. The apparatus of claim 18, wherein the processor is configured to determine a position and orientation of the imaging beam from the imager in response to the elongate probe pivoting about the opening.

20. The apparatus of claim 18, wherein the processor is configured to determine a position and an orientation of a tip of the probe from sensor data in response to the probe pivoting about the opening.

21-70. (canceled)
